(19) United States
(12) Patent Application Publication
(10) Pub. No.: US 2008/0076122 A1
(43) Pub. Date: Mar. 27, 2008

(54) Characterizing exposure to ionizing radiation

(75) Inventors: Andrew J. Wyrobek, Walnut Creek, CA (US); Matthew A. Coleman, Oakland, CA (US); David O. Nelson, Oakland, CA (US); James Tucker, Novi, MI (US)

Correspondence Address: Eddie E. Scott;Assistant Laboratory Counsel, Lawrence Livermore National Laboratory, L-703, P.O. Box 808, Livermore, CA, 94551, US

(73) Assignee: The Regents of the University of California

(21) Appl. No.: 11/528,296

(22) Filed: Sep. 26, 2006

(51) Int. Cl. C12Q 1/68; C12P 19/34; G06F 19/00

(52) U.S. Cl. 435/6; 435/91.2; 702/20

(57) ABSTRACT

A method of characterizing exposure to ionizing radiation, utilizing the steps of selecting a set of biomarker geness for characterizing exposure to ionizing radiation, and using the set of biomarker genes for characterizing exposure to ionizing radiation. The step of selecting a set of biomarker genes for characterizing exposure to ionizing radiation was developed utilizing a unique set of 420 oligonucleotide probes for human genes capable of discerning past exposure to different doses of ionizing radiation. The step of selecting a set of biomarker genes for characterizing exposure to ionizing radiation utilizes groupings of genes that represent candidate panels of mRNA biomarkers.

The United States Government has rights in this invention pursuant to Contract No. W-7405-ENG-48 between the United States Department of Energy and the University of California for the operation of Lawrence Livermore National Laboratory.

### BACKGROUND

1. Field of Endeavor

The present invention relates to ionizing radiation and more particularly to characterizing exposure to ionizing radiation.

2. State of Technology

U.S. Pat. No. 6,025,336 issued Feb. 15, 2000 to Kristin L. Goltry and Joel S. Greenberger provides the following state of technology information, “The potential consequences of exposure to ionizing radiation make a biological indicator of past radiation exposure highly desirable. But conventional approaches in this regard have limited the available evidence of past exposure largely to gross pathology or circumstantial evidence, such as telangiectasia (new vessel) formation, fibrosis of skin or other organs, alopecia, cataract formation, sterilization, and teratogenesis (birth defects) in subjects in the first trimester of pregnancy at the time of exposure. Molecular evidence of prior irradiation has been quite limited. For example, chromosome 2-specific deletions have been identified in hematopoietic cells of CBA/Ca mice that develop leukemia following exposure to an inducing dose of 200 cGy of ionizing radiation. The CBA/Ca mice develop a series of chromosomal abnormalities. While specific chromosomal changes reproducibly have been demonstrated in hematopoietic cells, there has been no report that gamma irradiation exerts other detectable effects on the hematopoietic stem cells either directly or indirectly, for example, through effects on cells of the bone marrow stromal microenvironment. Short-lived biochemical indicators also have been reported. For example, increased expression of mRNA for c-jun, c-fos and p53 has been observed in cell cultures following exposure to ionizing radiation. The increased expression is transient, with levels returning to normal as quickly as one hour after exposure. Similarly, elevated circulating levels of the protein TGF-.beta. have been observed in patients exposed to at least 3000 cGy of x-rays in radiotherapy. In patients that do not develop pneumonitis, this level returns to normal by the end of radiotherapy, while in most patients developing pneumonitis, the elevated levels persisted by the end of therapy. In addition, expression of TGF-.beta. varies from individual to individual. Both the brevity of the TGF-.beta. elevation and the variation in its expression from individual to individual renders the elevation unsuitable as an indicator of past exposure. Indeed, no biological indicator has been reported that is detectable weeks or months following exposure. A persistent, detectable indicator of past exposure to ionizing radiation would be valuable both in basic radiation biology and in forensic pathology. The need is evident particularly in circumstances where a prior history of radiation exposure is not suspected or is questioned as the etiological agent of a given pathological condition.”

### SUMMARY

Features and advantages of the present invention will become apparent from the following description. Applicants are providing this description, which includes drawings and examples of specific embodiments, to give a broad representation of the invention. Various changes and modifications within the spirit and scope of the invention will become apparent to those skilled in the art from this description and by practice of the invention. The scope of the invention is not intended to be limited to the particular forms disclosed and the invention covers all modifications, equivalents, and alternatives falling within the spirit and scope of the invention as defined by the claims.

Microarray transcript analysis methods were used to discover a unique set of 420 oligonucleotide probes for human genes capable of discerning past exposure to different doses of ionizing radiation. An individual's gene expression responses for the genes included in these panels of identified genes provides information on that person's prior exposure dose to ionizing radiation suitable for individual radiation biodosimetry. These panels of genes also have applications as clinical diagnostic of exposure to a variety of other genotoxic and pathogenic stresses. This is also relevant for estimating health effects related a wide range of IR exposures for cancer therapy.

The present invention utilizes the identification of groupings of human genes that represent candidate panels of mRNA biomarkers of generalized human exposure to ionizing radiation. These panels represent genes that are (a) dose specific over a range of 14 radiation doses including no exposure baseline data for these genes; (b) a robust panel that discriminate between low, moderate, and high-dose exposures; (c) a robust panel that is validated across multiple individuals.

The present invention provides a method of characterizing exposure to ionizing radiation, comprising the steps of selecting a set of biomarkers for characterizing exposure to ionizing radiation, and using said set of biomarkers for characterizing exposure to ionizing radiation. The step of selecting a set of biomarkers for characterizing exposure to ionizing radiation was developed producing a unique set of 420 oligonucleotide probes for human genes capable of discerning past exposure to different doses of ionizing radiation. The step of selecting a set of biomarkers for characterizing exposure to ionizing radiation comprises groupings of genes that represent candidate panels of mRNA biomarkers. In one embodiment the mRNA biomarkers represent genes that are dose specific over a range of 14 radiation doses including no exposure baseline data for these genes. In one embodiment the mRNA biomarkers represent groupings of genes that represent candidate panels of mRNA biomarkers that represent genes that are a robust panel that discriminate between low and high-dose exposures. In one embodiment the mRNA biomarkers represent groupings of genes that represent candidate panels of mRNA biomarkers that represent genes that are a robust panel that is validated across multiple individuals. In one embodiment the mRNA biomarkers represent groupings of genes that represent candidate panels of mRNA biomarkers that represent genes that are dose specific over a range of 14 radiation doses including no exposure baseline data for these genes; a robust panel that discriminate between low and high-dose exposures; or a robust panel that is validated across multiple individuals.

In one embodiment the step of using said set of biomarkers for characterizing exposure to ionizing radiation comprises using molecular techniques for characterizing exposure to ionizing radiation. In one embodiment the step of using said set of biomarkers for characterizing exposure to ionizing radiation comprises using PCR for characterizing exposure to ionizing radiation. In one embodiment the step of using said set of biomarkers for characterizing exposure to ionizing radiation comprises using DNA test strips for characterizing exposure to ionizing radiation. In one embodiment the step of using said set of biomarkers for characterizing exposure to ionizing radiation comprises using Luminex for characterizing exposure to ionizing radiation. In one embodiment the step of using said set of biomarkers for characterizing exposure to ionizing radiation comprises using microarrays for characterizing exposure to ionizing radiation. In one embodiment the step of using said set of biomarkers for characterizing exposure to ionizing radiation comprises using molecular techniques for characterizing exposure to ionizing radiation that are capable of measuring single or multiple combinations of transcript and protein changes for all or subset panels of the gene probes.

The present invention has use as an individualized radiation biodosimeter, for medical assessment to determine the appropriate treatment for an exposed individual, for triage after unexpected exposures (accident, terrorism and military), and for medical diagnostics including detection and identification of IR exposure and susceptibility to exposure.

The invention is susceptible to modifications and alternative forms. Specific embodiments are shown by way of example. It is to be understood that the invention is not limited to the particular forms disclosed. The invention covers all modifications, equivalents, and alternatives falling within the spirit and scope of the invention as defined by the claims.

### BRIEF DESCRIPTION OF THE DRAWINGS

The accompanying drawings, which are incorporated into and constitute a part of the specification, illustrate specific embodiments of the invention and, together with the general description of the invention given above, and the detailed description of the specific embodiments, serve to explain the principles of the invention.

FIG. 1. Selection of two sets of low-dose radiation responsive gene sets. The set 291 genes gave statistically significant differential expression for at least one low dose (1, 2.5, 5, 7.5 or 10 cGy) in either human cell line, GM15036 or GM14510. Up regulated genes had a FDR adjusted p-value at each dose of less that 0.05 and the lower limit of the confidence interval of the mean value of the expression (across all 5 doses low doses) greater than 1.0. Down-regulated genes had upper CI limits of less than 1.0. The genes that follow the above criteria and are consistent in both cell lines define the set of 81 consistent low dose sensitive genes.

FIG. 2. RT-PCR evaluation of the microarray results. The quantitative RT-PCR values (black bars) and microarray data (gray bars) are represented as means with 95% confidence intervals across the low dose regime (1-10 cGy). Seventeen genes were tested: CD164 (IMMUNE MODULATION), GGH (POSTTRANSLATIONAL PROCESSING), GLG1 (GOLGI APPARATUS PROTEIN), LIPA (LIPID METABOLISM), MAN1A2 (CARBOHYDRATE METABOLISM), PAM (OXIDOREDUCTASE ACTIVITY), PPT1 (PROTEIN-LIPID METABOLISM), SCAMP1 (INTRACELLULAR PROTEIN TRAFFIC), SLC38A1 (AMINO ACID TRANSPORT), SSR1 (MEMBRANE TRAFFIC), TRAM1 (INTRACELLULAR TRAFFIC), TMP21 (TRANSMEMBRANE TRAFFIC). All genes were normalized to GAPDH blevels within each cell line. The last five genes (FLJ10652, SLC38A1, HMMR, ACSL3, POLQ) were tested in cell line GM15036, and the first 12 in GM15510. The dotted line represents no radiation effect (i.e., unity fold change between irradiated and control samples).

FIG. 3. Dose response comparison for 81 gene set in two lymphoblastoid cell lines. Panel A. Slope analyses. Only one gene had a significant slope in both cell lines. Panel B. Intercept analyses. Sixteen genes had significant intercepts in both cell lines. Intersecting circles represent the genes with consistent responses in both cell lines. Numbers outside the circles represent the number of genes that do not have significant slopes (Panel A) or do not have significant intercepts (Panel B).

FIG. 4. Dose-response relationships for example genes in two cell lines. The fold change values of the genes vs. the dose (cGy) plotted for the cell lines GM15036 (left panels) and GM15510 (right panels). Triplicate expression values are shown at each dose. The dashed lines represent the linear regression lines. Genes GGH, HNRPD and TXNDC4 are representative examples of genes with large intercepts in both cell lines, while FLJ10618 is an example of the genes with slope as well as intercept values above the chosen cutoff in both cell lines.

FIG. 5. Composite model of gene interaction networks after low-dose cellular exposures. A composite model of Ingenuity-based gene networks within the 291 low dose (1-10 cGy) IR responsive gene set. The top 8 networks identified (Ingenuity rank scores>11) are labeled using Ingenuity functional areas. Shaded circles represent genes differentially expressed by low doses of IR. Open circles represent genes not identified within our experiments that are inferred because of known physical or functional associations with other genes. Lines indicate the connections between each gene. Rectangles indicate major nodes (TP53, MYC, FOS, BCL2, TBP, E2F1, EGR2 and TAFBP) with more that 5 associations. Gene names with asterisks indicate genes that were in common with the more stringent 81-gene set.

FIGS. 6, 7, and 8. Gene-interaction networks centered on TP53, MYC, and FOS functions. Panel 7: network model for TP53. Panel 8: network model for MYC. Panel 9: network model for FOS.

### DETAILED DESCRIPTION OF THE INVENTION

Referring to the drawings, to the following detailed description, and to incorporated materials, detailed information about the invention is provided including the description of specific embodiments. The detailed description serves to explain the principles of the invention. The invention is susceptible to modifications and alternative forms. The invention is not limited to the particular forms disclosed. The invention covers all modifications, equivalents, and alternatives falling within the spirit and scope of the invention as defined by the claims.

Ionizing radiation (IR) is a potent cell-killing and DNA-damaging agent commonly used in cancer therapy. High-dose exposures are known to lead to a variety of health effects such as tissue pathology, chromosome damage, life shortening and cancer; but little is known of the health effects associated with low dose. Current standards for occupational and residential exposure to IR are based on linear, no-threshold models of risk, even though there is epidemiological evidence that suggests the risks from exposure to low dose or low-dose rate IR may follow a non-linear dose-response relationship. Among Japanese A-bomb survivors, for example, severe mental retardation after uterine exposure and cancer incidences show non-linear effects below −0.2 Gy. Rapidly outpacing traditional single-gene approaches, microarray studies have shown IR-modulation of transcription factors, oncogenes, intercellular signaling factors and growth factors, as well as, genes involved in response to tissue injury, inflammation, oxidative stress, and protective responses. However, few of these studies were conducted using a single array type and the diversity of experimental conditions in multiple laboratories prevents meaningful comparisons to a broad spectrum of dose effects for correlating health effects. Our experiments provide a single platform for correlating IR dose and gene expression profiles.

There are currently no effective molecular biomarkers of radiation exposure. The general consensus is that individual biomarkers will be insufficient and that panels of molecular biomarkers will be needed. In most exposure scenarios the time of exposure will be known, but it will be difficult to control the time that individuals are evaluated after exposure. Based on prior experience with atomic bombs in Japan and accidental radiation exposure incidents, the vast majority of individuals who will need to be evaluated after an unexpected exposure will probably have received low doses that are medically insignificant.

The present invention provides a method of characterizing exposure to ionizing radiation, comprising the steps of selecting a set of biomarkers for characterizing exposure to ionizing radiation, and using said set of biomarkers for characterizing exposure to ionizing radiation. The step of selecting a set of biomarkers for characterizing exposure to ionizing radiation was developed utilizing a unique set of 420 oligonucleotide probes for human genes capable of discerning past exposure to different doses of ionizing radiation. The step of selecting a set of biomarkers for characterizing exposure to ionizing radiation comprises groupings of genes that represent candidate panels of mRNA biomarkers. In one embodiment the mRNA biomarkers represent genes that are dose specific over a range of 14 radiation doses including no exposure baseline data for these genes. In one embodiment the mRNA biomarkers represent groupings of genes that represent candidate panels of mRNA biomarkers that represent genes that are a robust panel that discriminate between low and high-dose exposures. In one embodiment the mRNA biomarkers represent groupings of genes that represent candidate panels of mRNA biomarkers that represent genes that are a robust panel that is validated across multiple individuals. In one embodiment the mRNA biomarkers represent groupings of genes that represent candidate panels of mRNA biomarkers that represent genes that are dose specific over a range of 14 radiation doses including no exposure baseline data for these genes; a robust panel that discriminate between low and high-dose exposures; or a robust panel that is validated across multiple individuals.

In one embodiment the step of using said set of biomarkers for characterizing exposure to ionizing radiation comprises using molecular techniques for characterizing exposure to ionizing radiation. In one embodiment the step of using said set of biomarkers for characterizing exposure to ionizing radiation comprises using PCR for characterizing exposure to ionizing radiation. In one embodiment the step of using said set of biomarkers for characterizing exposure to ionizing radiation comprises using DNA test strips for characterizing exposure to ionizing radiation. In one embodiment the step of using said set of biomarkers for characterizing exposure to ionizing radiation comprises using Luminex for characterizing exposure to ionizing radiation. In one embodiment the step of using said set of biomarkers for characterizing exposure to ionizing radiation comprises using microarrays for characterizing exposure to ionizing radiation. In one embodiment the step of using said set of biomarkers for characterizing exposure to ionizing radiation comprises using molecular techniques for characterizing exposure to ionizing radiation that are capable of measuring single or multiple combinations of transcript and protein changes for all or subset panels of the gene probes.

Transcript-Dose Responds and Cellular Networks Associated with Low-Dose Low-Let Ionizing Radiation in Human Lymphoblastoid Cells

Applicants have conducted studies, test, analysis, and other investigation in developing the present invention. Some of the studies, test, analysis, and other investigation will be described. The health consequences of high-dose ionizing radiation (IR) exposure are documented, but the health risks associated with low-dose exposures remain uncertain because of the paucity of epidemiological and molecular information at low doses. Applicants investigated the transcription profiles of two independent lymphoblastoid cell lines after IR exposures of 1, 2.5, 5, 7.5 and 10 cGy (¹³⁷Cs) using transcript microarrays containing over 22,000 probes to examine the effects of dose on transcript response, to identify novel low-dose radiation-responsive genes, and construct networks of low-dose cellular response functions. A candidate set of 291 low-dose responsive genes was identified (false discovery rate, <0.01) of which 81 genes showed consistent low-dose responses in both cell lines. Specific transcript responses were confirmed by real-time quantitative PCR. Regression analyses found that most IR-inducible genes did not have a significant slope consistent with plateau-like responses across the 1-10 cGy range. Applicants also identified several genes with significantly elevated (fold-change) expression at 1 cGy and significant Y-intercepts, indicating that the expression of these genes was modulated at doses below 1 cGy. Network analyses indicates that low-dose-responsive gene products are associated with cellular homeostasis (membrane signaling and damage sensing, small molecule transport, immune modulation, cell-cell communications, and cellular metabolism); signal transduction (linked to MYC, FOS and TP53 functions); and associated with various subcellular functions and locations (Golgi, mitochondria, and endoplasmic reticulum). The non-linear dose response characteristics of these low-dose genes and their broad biochemical and physical associations within the cell provide mechanistic insight for assessing tissue consequences and health risks of low-dose IR.

Tissue damage and health consequences after exposures to high-dose ionizing radiation (IR) are well documented, but considerable uncertainty remains about the health risks associated with exposures to low doses, i.e., <10 cGy. Human exposures to low dose ionizing radiation are by far more common than exposure to high-dose exposure, occurring from natural sources, cosmic rays, nuclear power and various sources of radioactive waste. Low-dose IR is also increasingly common in nuclear medicine, medical diagnostics, and dentistry. While there is substantial epidemiological evidence that doses in the range of 0.2 to 3.0 Sv increase the risks for cancer and other ill health effects, there are few epidemiological data for the consequences from lower doses resulting in considerable controversy on how to approach the problem of assessing health risks from low dose exposures.

The recent BIER VII report (2005) concluded that the available biological and biophysical data remain consistent with a “linear, no-threshold” (LNT) risk model, especially for solid tumors. This model predicts that the smallest doses of IR will increase the risk for tumors (2). In contrast, the recent report from the French Academy of Sciences questioned the application of the LNT model to low dose exposures in light of the mounting evidence for non-linearity in biochemical and cellular effects at low doses. Addressing the low-dose controversy is important because inappropriate application of empirical relationships for dose-risk relationships from higher dose data may lead to erroneous estimations of risk for lower dose exposures, which might discourage patients from undergoing useful medical examinations and may introduce bias in radioprotective measures against very low doses.

There has been considerable progress towards understanding the molecular and cellular responses after high and low IR exposures. High-dose exposures (>2 Sv) are known to modulate the expression of genes associated with genotoxic and physiological stress responses including cellular homeostasis, DNA damage sensing and repair, and immune response. At high doses, cellular decisions to initiate rescue or cell death appear to be mediated by various signaling pathways and secondary messengers. Several cytoplasmic pathways are rapidly activated after high-dose IR exposure, including cytoplasmic Ca²⁺ homeostasis mechanisms, kinase cascades and ceramide production. There are associated increases in mitochondrial permeability and release of the calcium. While DNA, and cellular membranes and organelles are important targets of radiation damage, their relative roles at low doses is poorly understood.

Transcriptional modulation is known to be very sensitive to IR exposure, especially after low doses. Several studies have characterized the genomic effects of low-dose IR on transcription for cells irradiated in vitro. Two studies have investigated transcript profiles after in vivo low dose IR exposures. Applicants' study of brain tissue from irradiated mice identified several hundred genes induced by 10 cGy, and showed that the 10 cGy transcript profile was qualitatively and quantitatively different from the 2 Gy profiles and differed over time after irradiation. Goldberg and colleagues identified five modulated genes in human skin irradiated at 1 cGy and doses above. These low-dose studies demonstrate that doses as low as 1 cGy (−0.4 direct ionizing tracks per cell) are sufficient to modulate the expression of a substantial number of genes. However, there is very little information on the shapes of low-dose effects on transcription profiles to assess their relevance to the LNT model.

The purpose of Applicants study was to identify sets of genes whose transcript expression was consistently modulated in the low-dose range, characterize the nature of the dose response curve in the low dose range (slope and intercept), and construct candidate networks of low-dose cellular response functions. Applicants investigated the transcription profiles of two independent human lymphoblastoid cell lines irradiated at 5 dose levels in the dose range of 1-10 cGy using microarrays containing 22,283 probes and a linear amplification procedure to identify low-abundance transcripts that are modulated upon exposure to low doses of IR.

Materials and Methods

Cell Culture, Irradiation, and RNA Extraction

Two human lymphoblastoid cell lines (GM15036 and GM15510) from the Coriell Cell Repositories, were grown in suspension in RPMI 1640 (Invitrogen, Carlsbad, Calif.) supplemented with heat inactivated 15% fetal bovine serum (Sigma), 1× antibiotic-antimycotic 100 units/ml penicillin G sodium, 100 μg/ml streptomycin, and 0.25 μg/ml amphotericin B as Fungizone® in 0.85% saline; and 2 mM L-glutamine (Invitrogen). Cultures were grown in a humidified 5% CO₂ atmosphere at 37° C., maintained at 1-10×10⁵ cells/ml. Ten ml of culture at 2-3×10⁵ cells/ml in T-25 flasks received fresh media 24 hours before irradiation. Approximately 5×10⁶ cells were irradiated using a ¹³⁷Cs Mark 1 Irradiator (J.L. Shepherd and Associates, Glendale, Calif.) to deliver 0 (sham) 1, 2.5, 5, 7.5 and, 10 cGy (dose rates: 1.5-4 cGy/min). Following irradiation, cells were incubated at 37° C. and harvested 4 hours after irradiation. Cultures were centrifuged, pellets washed with phosphate buffered saline, re-suspended in cold 1 ml of culture medium, transferred to a 2 mL cryo tube, flash frozen in liquid nitrogen, and stored at −80° C. Total RNA was extracted from thawed cells (RNeasy Total RNA Isolation Kit according to the manufacturer's protocol; QIAGEN, Valencia, Calif.) and treated with RNase-free DNase during the isolation procedure to remove contaminating genomic DNA (QIAGEN, Valencia, Calif.). RNA integrity and quantity were confirmed by agarose gel electrophoresis with ethidium bromide staining and spectrophotometry, or by using an Agilent 2100 Bioanalyzer and the RNA 6000 Nano Assay LabChip. Purified total RNA was stored at −80° C. until further use. This study consisted of 12 biological samples (2 cell lines×6 doses including SHAM control).

Microarray Analyses

Total RNA from each sample was amplified using the ARCTURUS RiboAmp® kit (ARCTURUS, Mountainview, Calif.). The processed RNA was then converted to double stranded cDNA and the Enzo BioArray HighYield Transcript Kit was used for RNA amplification-based labeling (Enzo Biochem, New York, N.Y.). After labeling, the antisense RNA was fragmented as described in the Affymetrix Gene Expression Analysis Technical Manual (Affymetrix, Santa Clara, Calif.) and evaluated for quality and quantity on the Agilent 2100 Bioanalyzer prior to microarray hybridization. Thirty six Affymetrix HG-U133A gene chips (3 triplicates per sample) were hybridized using 15 μg of fragmented complementary RNA followed by washing and staining in an Affymetrix Fluidics Workstation as described in the Expression Analysis Technical Manual (Affymetrix, Santa Clara, Calif.). Hybridized chips were scanned and signals detected using an argon-ion laser scanner (Agilent Technologies, Palo Alto, Calif.). Microarray reports were generated to assess the hybridization quality and individual CEL files were used for data preprocessing as described below.

Statistical Analyses

Selection of “candidate” and “consistent” sets of low-dose responsive genes: Fold changes for each gene were calculated across doses by fitting a simple linear model to the data, using 30 degrees of freedom (36 chips—6 doses) to estimate the noise. The p values generated by MAS-5 were used to identify genes with a significant hybridization signals, and were adjusted by the Benjamini-Hochberg method to control the per-chip false discovery rate, using the “mt.rawp2adjp” procedure in Bioconductor). Only genes with an FDR-adjusted p value not exceeding 0.01 were selected. For each gene and cell line, t-statistics were computed for each log fold change. QQ-plots were used to compare the distribution of the t-statistics. Robust linear regression was used on the observed versus theoretical quantiles to determine what linear transformation of these t-statistics would confer a normal distribution, and then scaled accordingly. The per-chip expression data corresponding to genes with a positive signal were combined in a two-step process to obtain an initial analytical data set. First, the initial data set consisted of expression data for all the genes for which a signal was detected at least one dose for that cell line. Second, the data from the two cell lines were combined into a single data set. The resulting data set consisted of genes that had expression in one or both cell lines for at least one of the doses tested. Differential expression across the six IR doses was detected by an F test, and a separate F test was performed on each gene and each cell line. After adjusting the p values for these genes using the Benjamini-Hochberg procedure, 291 genes (referred to as the “candidate set” of low-dose responsive genes) showed an adjusted F test p value of <=0.05 indicating differential expression across the low dose range. Table S1 (supporting material) contains the complete list of the 291 genes, with p values and fold changes in both cell lines. Using the confidence interval (CI) of the average fold change across the doses tested for all 291 genes, Applicants identified up-regulated genes that had lower limit of CI>1.00, while those with an upper limit of the CI<1.00 were designated as “down regulated.” A total of 81 genes showed common responses in both cell lines (referred to as the “consistent set” of low dose responsive genes).

Dose response analyses (Intercept and slope): The expression profiles of the 81 genes representing the common-response for both cell lines were evaluated by linear regression analyses, using the Linear Models for Microarray Data (LIMMA) package in the R statistical environment. A language for data analysis and graphics: J. Comput. Graph. Stat. 5 299-314. 1996.). LIMMA was used to determine the fit of the linear model, to obtain the 95% CI on the sham irradiated samples and to determine the slope and intercept values with 95% CI the regression of the 5 dose values for fold change for each gene for each cell line.

Pathways and Network Analysis

Gene annotations and functional classifications were assessed with EASE and the “Gene Ontology” (GO) database, with screening for statistically overrepresented categories using GOstat.org by Beisbarth. Gene network and pathway analyses were performed using Ingenuity Systems Pathway Knowledge Database). Genes from the two input lists (i.e., candidate and consistent gene lists) were mapped to distinct biological networks. The rank score for each pathway is the probability that a collection of genes equal to or greater that the number in a network could be achieved by chance alone. Analyses was limited to those networks with rank scores of >3 (i.e., >99.9% chance of not being generated by random chance) and Fischer exact p-values were used to determine the probability that a biological function assigned to a network occurred by chance. Applicants found 274 unique annotated genes within the 291-gene set. Similarly, there were 75 unique annotated genes in the 81-gene set. Others radiation-responsive probe sets may be detecting alternatively spliced products or redundant transcripts.

RT-PCR Validation

Seventeen genes were selected based on an intensity filter (>500 signal intensity values and >1.5 average fold changes across doses) for evaluation by quantitative real-time reverse transcriptase PCR (QRT-PCR). First, 1-2 micrograms of total RNA and random primers were used to generate cDNA (High Capacity Archive Kit, Applied Biosystems, Inc., Foster City Calif.). Second, aliquots of cDNA were used to perform PCR using TaqMan® Gene Expression Assays, with gene specific primers (900 nM each) and fluorescent probes. These primers were specifically designed to span intronic regions of the respective genes of interest. The reactions were run on an AB Sequence Detection System 7900HT Fast Real-Time PCR System using a 384 well format, with both no-template and no-primer controls. The reaction conditions were as follows: (step 1) 95° C. for 1 minute, (step 2) 95° C. for 15 seconds, (step 3) 60° C. for 30 seconds, (step 4) cycle through steps 2 and 3 for an additional 39 times, and a final hold at 4° C. Efficiency of amplification for all primer probe sets was estimated to be >96% (Data not shown). Multiple endogenous controls including 18S, microglobulin, actin and GAPDH were analyzed (data not shown); GAPDH was determined to be the least variant and hence used for relative quantification with GAPDH as endogenous control to generate the delta Ct values, and fold changes in gene expression. The normalized results for each of the four replicates of each tested gene were averaged and compared to unity and microarray fold changes, using confidence intervals (95% CI). The following gene-specific assays were used: CD164 (Hs00174789_m1), GGH (Hs00608257_m1), GLG1 (Hs00201886_m1), LIPA (Hs00426932_m1), MAN1A2 (Hs00198611_m1), PAM (Hs00168596_m1), PPT1 (Hs00165579_m1), SCAMP1 (Hs00792736_m1), SLC38A1 (Hs00229126_m1), SSR1 (Hs00162340_m1), TRAM1 (Hs00560089_m1), MP21 (Hs00828975_s1).

Microarray Identification of Low-Dose Responsive Genes and RT-PCR Verifications

Analyses of the gene-transcript microarray data yielded a “candidate” set of 291 genes that showed transcript modulation in at least one of the two independent cell lines (GM15036 and GM15510) for one or more of the 5 doses tested: 1, 2.5, 5.0, 7.5, 10 cGy.

TABLE 1

| | | | Line GM15036 | | | | Line GM15510 | | | |
|---|---|---|---|---|---|---|---|---|---|---|
| | | | Fold- | | | | Fold- | | | |
| | SYMBOL | ACCNUM | change | CI-low | CI-high | p-value | change | CI-low | CI-high | p-value |
| 1 | AP311304 | NM_031214 | 2.0 | 1.7 | 2.4 | 0.042 | 2.2 | 2.8 | 2.7 | 0.003 |
| 2 | ALG5 | NM_G13338 | 1.2 | 1.5 | 2.0 | 0.001 | 1.8 | 1.6 | 1.9 | 0.000 |
| 3 | ATP10D | AI47B147 | 2.3 | 1.7 | 3.0 | 0.018 | 2.2 | 1.8 | 2.5 | 0.004 |
| 4 | ATP1B3 | U51478 | 1.5 | 1.3 | 2. | 0.000 | 1.5 | 1.3 | 1.7 | 0.000 |
| 5 | BTAP1 | AJ001017 | 1.7 | 1.4 | 1.9 | 0.020 | 1.4 | 1.3 | 1.5 | 0.019 |
| 6 | C11 | NM_020644 | 1.6 | 1.5 | 1.5 | 0.010 | 1.5 | 1.4 | 1.6 | 0.004 |
| 7 | C | NM_004872 | 2.1 | 1.6 | 2.6 | 0.011 | 1.6 | 1.5 | 1.7 | 0.003 |
| 8 | CD164 | NM_006016 | 1.7 | 1.4 | 2.1 | 0.029 | 1.9 | 1.7 | 2.0 | 0.000 |
| 9 | CD43 | NM_001778 | 1.6 | 1.4 | 1.8 | 0.003 | 1.5 | 1.4 | 1.7 | 0.036 |
| 10 | CD53 | NM_000560 | 1.4 | 1.3 | 1.6 | 0.012 | 1.4 | 13 | 1.5 | 0.003 |
| 11 | CD53 | BC005930 | 2.5 | 1.8 | 3.1 | 0.000 | 1.9 | 1.8 | 2.1 | 0.000 |
| 12 | CD53 | NM_001779 | 2.0 | 1.6 | 1.5 | 0.000 | 1.6 | 1.4 | 1.7 | 0.000 |
| 13 | CD53 | D28586 | 2.2 | 1.5 | 3.9 | 0.01 | 1.7 | 1.5 | 2.0 | 0.022 |
| 14 | CDI-100 | AL117354 | 2.1 | 1.5 | 3.7 | 0.010 | 1.5 | 1.4 | 1.7 | 0.004 |
| 15 | CKI | NM_017801 | 1.7 | 1.5 | 1.9 | 0.001 | 1.6 | 1.4 | 1.7 | 0.004 |
| 16 | CNTH | NM_005776 | 1. | 1.4 | 2.2 | 0.041 | 1.5 | 1.4 | 1.6 | 0.016 |
| 17 | COCH | AA669336 | 1.6 | 1.4 | 1.9 | 0.019 | 1.5 | 1.4 | 1.6 | 0.013 |
| 18 | DJ971N18.2 | BP572868 | 2.2 | 1.7 | 2.5 | 0.035 | 2.0 | 1.3 | 2.1 | 0.002 |
| 19 | DSG2 | BP031829 | 2.3 | 1.7 | 2.9 | 0.032 | 2.0 | 1.8 | 2.0 | 0.001 |
| 20 | ELOVLS | AL136939 | 2.1 | 1.7 | 2.4 | 0.000 | 1.7 | 1.5 | 1.9 | 0.000 |
| 21 | ENTD1 | US7967 | 1.8 | 1.4 | 2.2 | 0.044 | 1.9 | 1.7 | 2.1 | 0.000 |
| 22 | EPRS | NM_004446 | 1.6 | 1.3 | 1.9 | 0.044 | 1.4 | 1.3 | 1.5 | 0.005 |
| 23 | FACL3 | AL535798 | 1.6 | 1.4 | 1.7 | 0.043 | 1.6 | 1.5 | 1.7 | 0.007 |
| 24 | FL310652 | NM_018169 | 1.8 | 1.5 | 2.1 | 0.014 | 1.7 | 1.4 | 2.0 | 0.011 |
| 25 | FL310900 | NM_018264 | 1.7 | 1.4 | 1.9 | 0.042 | 1.7 | 1.5 | 2.0 | 0.005 |
| 26 | GALNT | NM_017423 | 1.8 | 1.4 | 2.2 | 0.042 | 1.8 | 1.7 | 2.0 | 0.001 |
| 27 | GGH | NM_003878 | 2.5 | 1.8 | 3.2 | 0.001 | 1.9 | 1.8 | 2.1 | 0.000 |
| 28 | GLG1 | AK025457 | 1.8 | 1.5 | 2.2 | 0.025 | 1.9 | 1.7 | 2.1 | 0.000 |
| 29 | GMH1 | NM_014044 | 2.4 | 2.0 | 2.8 | 0.000 | 1.8 | 1.6 | 2.0 | 0.001 |
| 30 | HIP14 | AI621223 | 1.7 | 1.6 | 1.9 | 0.001 | 1.5 | 1.4 | 1.7 | 0.004 |
| 31 | HLA-B | D83043 | 1.7 | 1.4 | 1.9 | 0.010 | 1.5 | 1.3 | 1.7 | 0.011 |
| 32 | HMGCR | AL518627 | 2.1 | 1.7 | 2.5 | 0.011 | 1.8 | 1.5 | 2.1 | 0.047 |
| 33 | HMMR | NM_012485 | 2.0 | 1.6 | 2.3 | 0.047 | 1.6 | 1.4 | 1.7 | 0.036 |
| 34 | HNRPD | W$$ | 3.1 | 2.4 | 3.3 | 0.000 | 2.0 | 1.8 | 2.3 | 0.000 |
| 35 | HTGN29 | NM_020199 | 1.7 | 2.4 | 2.0 | 0.014 | 1.5 | 1.0 | 1.6 | 0.002 |
| 36 | IJ44 | BE049439 | 2.2 | 1.7 | 2.6 | 0.016 | 1.9 | 1.7 | 2.1 | 0.002 |
| 37 | JWA | NM_006407 | 1.7 | 1.4 | 1.9 | 0.005 | 1.6 | 1.5 | 1.7 | 0.001 |
| 38 | KIAA0102 | BP530535 | 2.1 | 2.7 | 2.4 | 0.000 | 1.6 | 1.6 | 1.7 | 0.000 |
| 39 | KTN1 | Z22551 | 1.3 | 1.4 | 1.7 | 0.043 | 1.6 | 1.4 | 1.8 | 0.023 |
| 40 | LIPA | NM_000235 | 2.2 | 1.7 | 2.7 | 0.001 | 2.0 | 1.9 | 2.2 | 0.000 |
| 41 | LOC5635 | NM_020154 | 2.2 | 1.6 | 2.7 | 0.000 | 1.8 | 1.6 | 2.0 | 0.000 |
| 42 | LYRIC | AI972475 | 1.4 | 1.3 | 1.5 | 0.012 | 1.3 | 1.2 | 1.5 | 0.049 |
| 43 | MAN1A1 | 6G287153 | 2.0 | 1.5 | 2.5 | 0.033 | 1.8 | 1.5 | 2.1 | 0.036 |
| 44 | MGC5306 | NM_024115 | 2.1 | 1.7 | 2.5 | 0.006 | 2.0 | 1.8 | 2.2 | 0.000 |
| 45 | MGC8721 | NM_016127 | 2.0 | 1.7 | 2.2 | 0.000 | 1.6 | 1.4 | 1.7 | 0.001 |
| 46 | MS4A1 | 5C002607 | 1.7 | 1.4 | 2.0 | 0.025 | 1.5 | 1.4 | 1.7 | 0.037 |
| 47 | P5 | AK026926 | 2.1 | 1.7 | 2.5 | 0.000 | 1.6 | 1.3 | 1.8 | 0.001 |
| 48 | P5 | NM_005742 | 1.5 | 1.3 | 1.6 | 0.013 | 1.5 | 1.3 | 1.7 | 0.013 |
| 49 | P5 | SC001312 | 1.5 | 1.3 | 1.6 | 0.018 | 1.4 | 1.3 | 1.6 | 0.011 |
| 50 | PAM | NM_000919 | 1.5 | 1.4 | 1.7 | 0.044 | 1.5 | 1.4 | 1.6 | 0.008 |
| 51 | POLQ | NM_014125 | 1.9 | 1.6 | 2.2 | 0.039 | 1.6 | 1.6 | 1.6 | 0.005 |
| 52 | PRDX4 | NM_006406 | 1.8 | 1.5 | 2.1 | 0.004 | 1.9 | 1.6 | 2.1 | 0.000 |
| 53 | PTPRC | Y00062 | 2.3 | 1.8 | 2.7 | 0.010 | 1.7 | 1.6 | 1.9 | 0.013 |
| 54 | PTTGIIP | NM_004339 | 1.6 | 1.4 | 1.9 | 0.003 | 1.4 | 1.3 | 1.5 | 0.004 |
| 55 | RNASET2 | NM_003730 | 1.7 | 1.5 | 1.9 | 0.010 | 1.6 | 1.4 | 1.7 | 0.005 |
| 56 | RNASET2 | NM_003730 | 1.4 | 1.3 | 1.5 | 0.047 | 1.6 | 1.4 | 1.6 | 0.003 |
| 57 | RPL5 | AL137958 | 1.9 | 1.4 | 2.3 | 0.034 | 1.8 | 1.7 | 1.9 | 0.000 |
| 58 | RPN2 | 5C003560 | 1.8 | 1.5 | 2.2 | 0.013 | 1.6 | 1.5 | 1.7 | 0.001 |
| 59 | SART2 | NM_013362 | 1.9 | 1.7 | 2.2 | 0.002 | 1.8 | 1.6 | 2.0 | 0.001 |
| 60 | SCAMP1 | AV745949 | 1.8 | 1.5 | 2.1 | 0.015 | 2.0 | 1.8 | 2.3 | 0.000 |
| 61 | SLC1A1 | AW235061 | 1.8 | 1.4 | 2.1 | 0.011 | 1.7 | 1.6 | 1.8 | 0.001 |
| 62 | SLC30A1 | AI972416 | 2.0 | 1.6 | 2.4 | 0.004 | 2.0 | 1.7 | 2.3 | 0.000 |
| 63 | SLC35A3 | NM_012243 | 1.8 | 1.6 | 2.1 | 0.046 | 1.9 | 1.6 | 2.1 | 0.028 |
| 64 | SLC38A1 | NM_030674 | 1.8 | 1.6 | 2.1 | 0.001 | 1.5 | 1.4 | 1.6 | 0.005 |
| 65 | SLC39A6 | AI635449 | 1.9 | 1.6 | 2.2 | 0.001 | 1.8 | 1.6 | 2.1 | 0.000 |
| 66 | SMBP | NM_020123 | 1.6 | 1.4 | 1.8 | 0.043 | 1.6 | 1.4 | 1.8 | 0.010 |
| 67 | SORL1 | AV728268 | 1.7 | 1.5 | 2.0 | 0.009 | 1.6 | 1.5 | 1.8 | 0.001 |
| 68 | SPTLC1 | AL558804 | 2.1 | 1.7 | 2.5 | 0.011 | 2.0 | 1.8 | 2.3 | 0.003 |
| 69 | SOLE | AF198865 | 2.0 | 1.6 | 2.4 | 0.021 | 1.9 | 1.5 | 2.2 | 0.015 |
| 70 | SSR1 | AI016620 | 1.7 | 1.4 | 2.1 | 0.006 | 2.0 | 1.7 | 2.2 | 0.000 |
| 71 | TEB4 | SF100409 | 1.5 | 1.4 | 1.6 | 0.005 | 1.6 | 1.4 | 1.8 | 0.001 |
| 72 | TFRC | NM_003234 | 2.5 | 1.9 | 3.2 | 0.000 | 1.6 | 1.4 | 1.7 | 0.000 |
| 73 | TGOLN2 | W72053 | 1.5 | 1.4 | 1.6 | 0.037 | 1.4 | 1.3 | 1.5 | 0.015 |
| 74 | TLOC1 | U93239 | 1.8 | 1.5 | 2.0 | 0.000 | 1.6 | 1.4 | 1.8 | 0.000 |
| 75 | TM9SF2 | NM_004800 | 2.6 | 1.8 | 3.3 | 0.002 | 2.2 | 1.9 | 2.4 | 0.000 |
| 76 | TMP21 | 5E780075 | 1.9 | 1.5 | 2.3 | 0.020 | 1.9 | 1.6 | 2.1 | 0.001 |
| 77 | TNFRSF6 | AA164751 | 2.0 | 1.5 | 2.6 | 0.003 | 1.3 | 1.5 | 2.0 | 0.009 |
| 78 | TRAM1 | 5C000687 | 1.8 | 1.4 | 2.2 | 0.015 | 2.0 | 1.9 | 2.1 | 0.000 |
| 79 | TXNDC4 | 5C005374 | 2.5 | 1.9 | 3.1 | 0.000 | 2.1 | 1.8 | 2.4 | 0.000 |
| 80 | VMP1 | NM_030938 | 1.7 | 1.4 | 1.9 | 0.000 | 1.4 | 1.3 | 1.5 | 0.000 |
| 81 | ZMPSTE24 | NM_005857 | 1.8 | 1.4 | 2.1 | 0.010 | 1.7 | 1.6 | 1.9 | 0.000 |

a) Average of fold Change across 5 low doses each with three technical replicares (15 values)
b) Upper and lower Confidence interval (95%) around the average fold change
c) FDR (False Discovery Rate) adjusted p value for multiple comparison

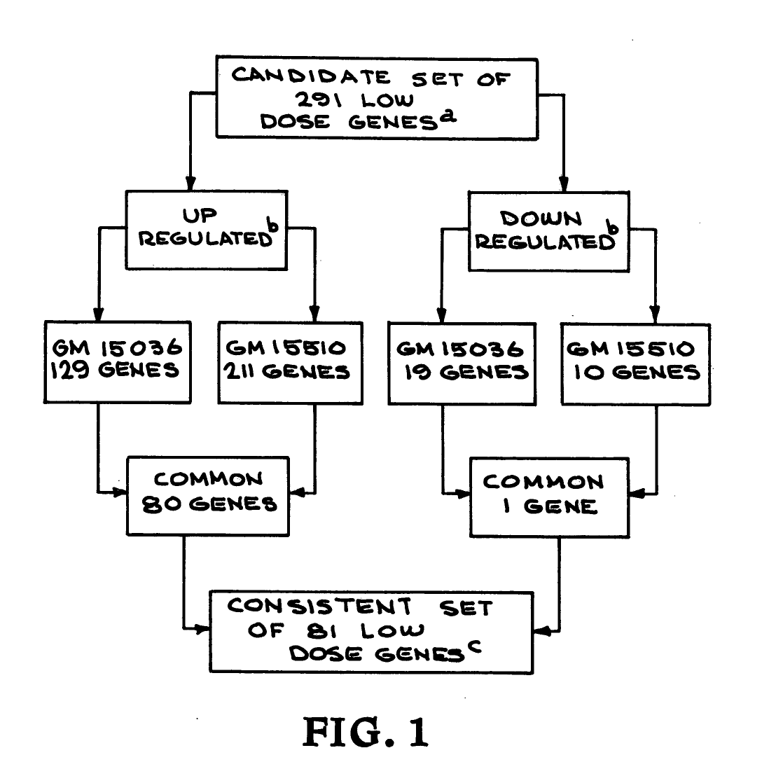

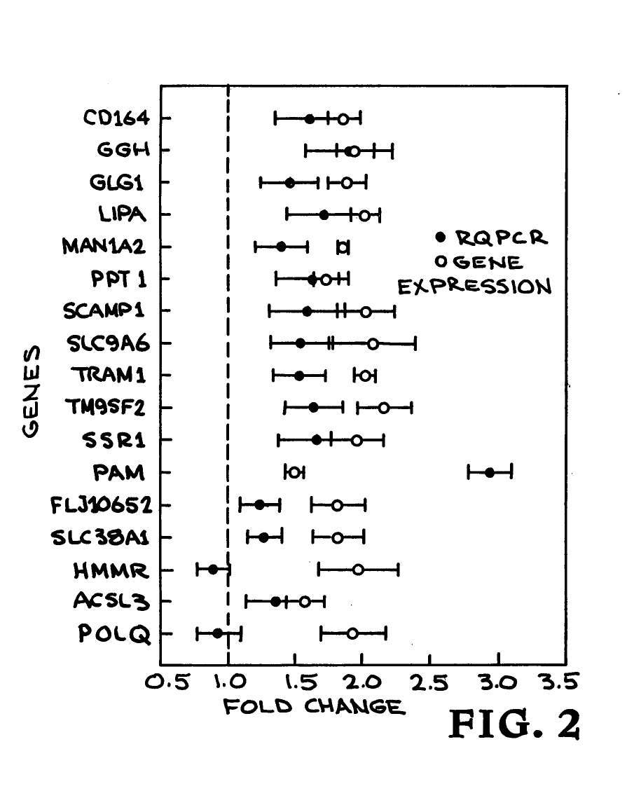

Applicants then selected genes that met the following two criteria: (1) more than 1.5 average fold increase across all doses tested and (2) a 95% confidence interval that excluded a fold change of unity. This process (FIG. 1) identified 129 and 211 up-regulated genes, and 19 and 10 down-regulated genes for GM15036 and GM15510, respectively. A set of 81 genes showed consistent responses in both cell lines (“consistent” set) of which 80 were up-regulated and one was down-regulated (FIG. 1, Table 1—Set of 81 genes with consistent low-dose response in lymphoblastoid cell lines from two individuals (in alphabetical order). Approximately 10% of these genes had a >2.2 average fold change after IR exposure (Table 2—Distribution of radiation-induced fold changes across the 81-gene set in two lymphoblastoid cell lines). A subset of 17 genes from the consistent set that had >500 normalized microarray intensity units and with >1.5 fold average increase after radiation in both cell lines was selected for evaluation using quantitative RT-PCR (QRT-PCR). The QRT-PCR results (FIG. 2) confirmed the low-dose radiation-induction for 15 of the 17 genes.

TABLE 2

| | Line GM15036 | Line GM15510 |
|---|---|---|
| Fold Change | No. of genes | No. of genes |
| >1.8 | 42 | 30 |
| >2.0 | 27 | 9 |
| >2.2 | 11 | 0 |

Dose Response and Intercept Analysis

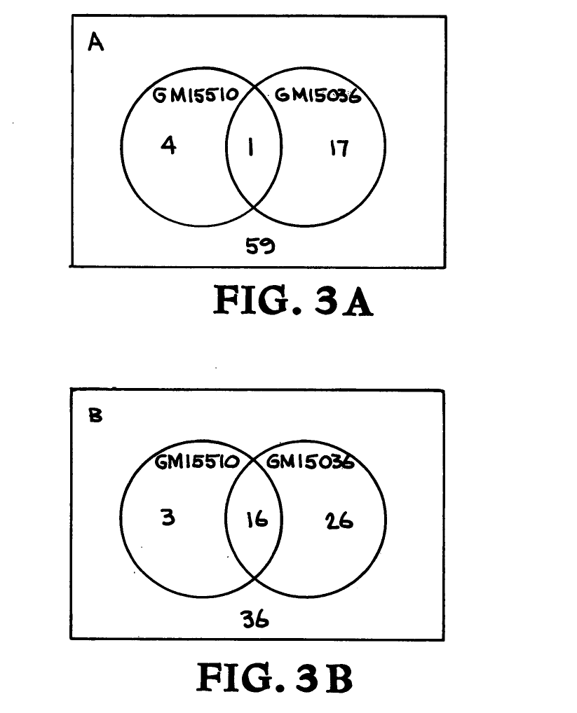

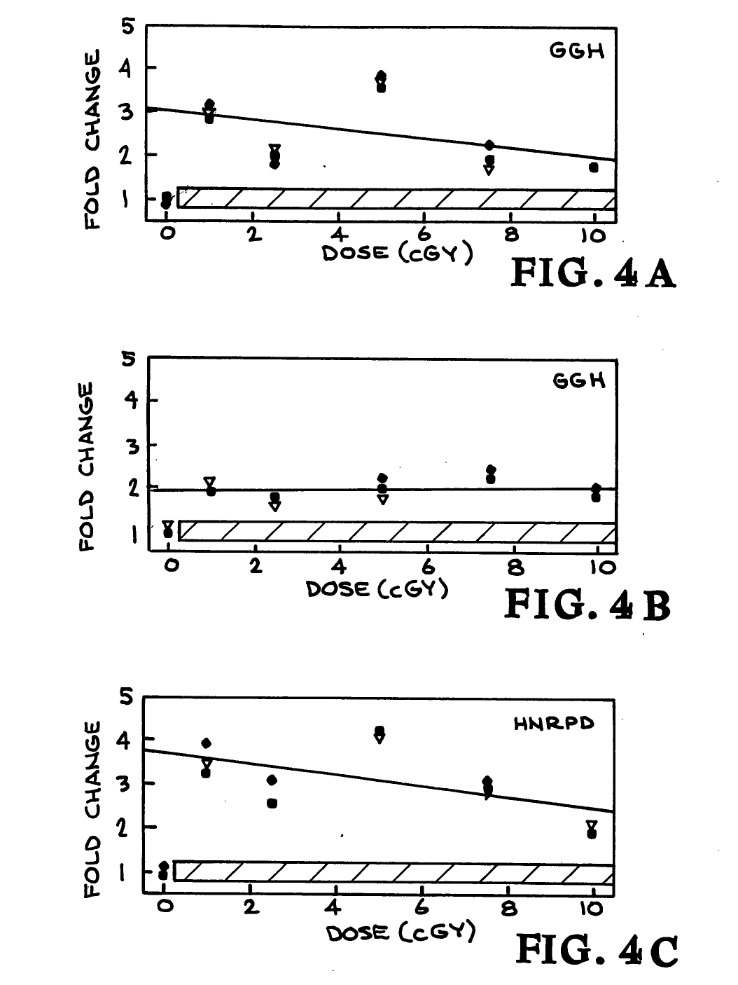

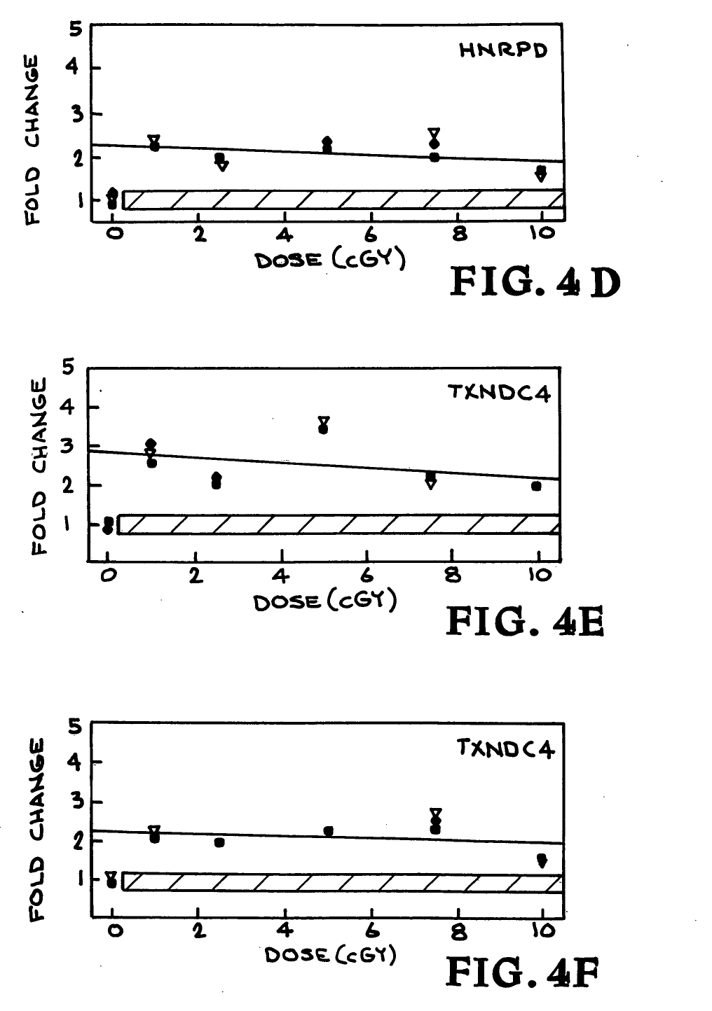

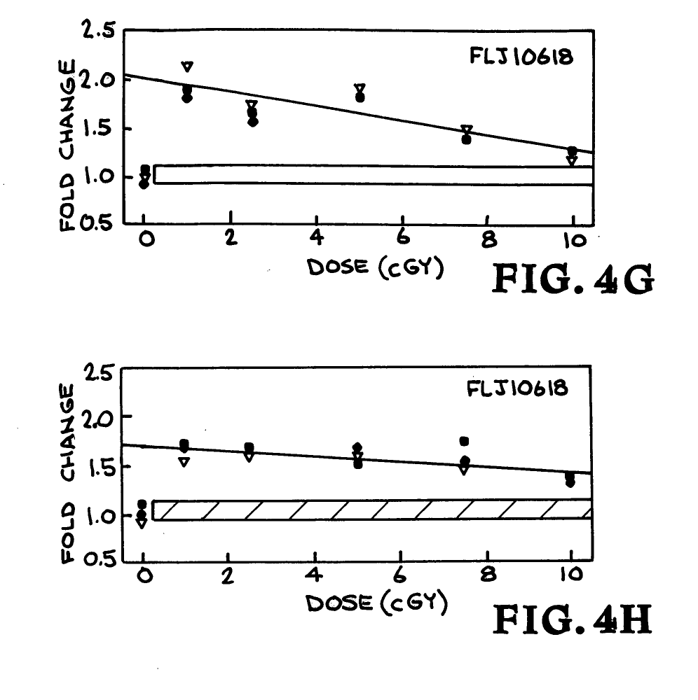

The shapes of the low-dose responses (slope and Y-intercepts) were analyzed for the set of 81 consistent genes by linear regression analyses of transcript expression in the 1-10 cGy range for both cell lines. Using linear regression analyses of normalized microarray intensity values only 1 gene showed significant slopes in both cell lines (FIG. 3A, Thioredoxin domain containing protein 13, DJ971N18.2) with slopes (95% CI) of 1.8 (1.3:2.3) and 1.8 (1.4:2.1) for cell lines GM15510 and GM15036, respectively (p<2E-10). Among the 81 genes, 22 genes had a statistically significant slope in either one or the other cell line, all of which increased with dose except for H2AFX, which decreased with dose. The majority of genes did not have a significant slope in either cell line (i.e., showed consistently flat responses with dose). Based on the linear analyses of fold changes in relation to unexposed cultures (FIG. 3B), 16 genes had consistently elevated intercepts in both cell lines, ranging from 2.6 to 3.5 fold changes for cell line GM15036, and 1.7 to 2.6 fold-changes for cell line GM15510, (Table 3—Genes with significant fold-change intercepts in lymphoblastoid cell lines from two individuals). The majority of genes (45) had significant intercepts in only one cell line (FIG. 3B), and the intercepts for the remaining 36 genes were not significantly different from unity. Consistent with these findings, many genes had significant elevated fold changes at 1 cGy (data not shown). FIG. 4 illustrates the dose response results for example genes from both cell lines.

TABLE 3

| | GM15036 | | | | | | GM15510 | | | | | |
|---|---|---|---|---|---|---|---|---|---|---|---|---|
| | SHAM^{a} | | | | | | SHAM^{a} | | | | | |
| | CI- | CI- | Low Dose Irradiation^{b} | | | | | CI- | Low Dose Irradiation^{b} | | | |
| Genes | low | high | Intercept | P-value | CI-low | CI-high | CI-low | high | Intercept | P-value | CI-low | CI-high |
| P5 | 0.98 | 1.02 | 2.38 | 3.78E−13 | 1.79 | 2.97 | 0.90 | 1.10 | 2.65 | 1.33E−13 | 2.05 | 3.25 |
| SLC30A1 | 0.94 | 1.06 | 2.32 | 5.29E−15 | 1.50 | 3.14 | 0.87 | 1.13 | 2.44 | 2.66E−15 | 1.58 | 3.31 |
| ATP10D | 0.96 | 1.04 | 2.81 | 1.09E−13 | 2.16 | 3.46 | 0.88 | 1.12 | 2.40 | 3.64E−14 | 1.73 | 3.06 |
| SCAMP1 | 0.91 | 1.09 | 2.07 | 3.04E−15 | 1.20 | 2.93 | 0.88 | 1.12 | 2.35 | 3.76E−13 | 1.79 | 2.91 |
| DSG2 | 0.86 | 1.14 | 2.54 | 4.34E−16 | 1.46 | 3.61 | 0.86 | 1.14 | 2.35 | 4.85E−13 | 1.80 | 2.89 |
| TM9SF2 | 0.91 | 1.09 | 3.01 | 1.31E−14 | 2.25 | 3.77 | 0.95 | 1.05 | 2.26 | 1.80E−14 | 1.56 | 2.96 |
| SQLE | 0.90 | 1.10 | 2.44 | 2.58E−15 | 1.57 | 3.32 | 0.93 | 1.07 | 2.17 | 5.05E−14 | 1.52 | 2.82 |
| DJ971N18.2 | 0.78 | 1.22 | 2.69 | 6.48E−13 | 2.12 | 3.25 | 0.88 | 1.12 | 2.16 | 1.53E−13 | 1.57 | 2.76 |
| HMGCR | 0.94 | 1.06 | 2.41 | 1.71E−13 | 1.79 | 3.04 | 0.96 | 1.04 | 2.12 | 1.75E−11 | 1.69 | 2.55 |
| HNRPD | 0.93 | 1.07 | 3.52 | 4.21E−15 | 2.68 | 4.36 | 0.88 | 1.12 | 2.10 | 6.05E−15 | 1.32 | 2.88 |
| GGH | 0.92 | 1.08 | 2.98 | 6.91E−16 | 1.99 | 3.96 | 0.90 | 1.10 | 2.07 | 4.89E−15 | 1.27 | 2.87 |
| GALNT7 | 0.90 | 1.10 | 2.16 | 1.30E−14 | 1.40 | 2.92 | 0.90 | 1.10 | 2.06 | 1.78E−14 | 1.35 | 2.76 |
| SPTLC1 | 0.84 | 1.16 | 2.12 | 2.22E−14 | 1.39 | 2.85 | 0.70 | 1.30 | 2.04 | 4.80E−14 | 1.39 | 2.69 |
| TMP21 | 0.87 | 1.13 | 2.15 | 1.51E−15 | 1.24 | 3.06 | 0.90 | 1.10 | 1.86 | 6.00E−14 | 1.22 | 2.49 |
| CD58 | 0.92 | 1.08 | 2.33 | 4.78E−16 | 1.27 | 3.40 | 0.90 | 1.10 | 1.72 | 1.19E−11 | 1.28 | 2.16 |
| MAN1A1 | 0.94 | 1.06 | 2.21 | 8.70E−13 | 1.65 | 2.76 | 0.89 | 1.11 | 1.72 | 1.18E−12 | 1.20 | 2.23 |

Bioinformatic Analyses of the Cellular Location and Function of the Low Dose Genes

Both the candidate and consistent sets of low-dose responsive genes were assigned to putative biological process and cellular location using the Gene Ontology (GO) database, which provided information for ˜70% of genes in Applicants data sets (Table 4—Gene Ontology (GO) assignments of the low dose genes to biological process and cell location for the 291 and 81 gene sets.). For biological processes, significant assignments were obtained for macromolecular metabolism, cell growth and/or maintenance, biosynthesis, and lipid metabolism (range of p values, 0.006<p<0.08). For cellular location, significant assignments were obtained for integral to membrane, cytoplasm, endoplasmic system, plasma membrane, membrane fraction, and soluble fraction (5E-9<p<0.04). Functional analyses using EASE were consistent the GO-based assignments for the following functions: cell-to-cell signaling and interaction (range of Fisher exact values, 1.51E-3 to 4.82E-2), cellular and organelle membrane structure and assembly (2.33E-4 to 4.82E-2), cell proliferation and cell cycle (6.48E-4 to 4.82E-2), DNA replication, recombination and repair (4.92E-3 to 2.92E-2), immune system modulation (8.30E-4 to 4.35E-2), lipid metabolism (4.92E-3 to 4.35E-2), cell death (2.38E-3 to 4.82E-2) and cancer (2.33E-4 to 4.82E-2).

TABLE 4

| | | 291 gene set | | | 81 gene set | | |
|---|---|---|---|---|---|---|---|
| Category | Terms | Count (a) | % (b) | p-value (c) | Count (a) | % (b) | p-value (c) |
| Biological | MACROMOLECULE | 26 | 31.2 | 6.48E−03 | 23 | 31.6 | 4.83E−03 |
| Process | METABOLISM | | | | | | |
| | CELL GROWTH AND/OR | 26 | 31.2 | 2.98E−02 | 23 | 31.6 | 2.41E−02 |
| | MAINTENANCE | | | | | | |
| | BIOSYNTHESIS | 10 | 13 | 4.17E−02 | 10 | 12.7 | 5.03E−02 |
| | LIPID METABOLISM | 6 | 7.8 | 7.48E−02 | 6 | 7.6 | 8.39E−02 |
| Cellular | INTEGRAL TO MEMBRANE | 46 | 59.7 | 5.56E−09 | 46 | 58.2 | 3.29E−08 |
| Location | CYTOPLASM | 37 | 42.9 | 1.41E−03 | 34 | 43 | 1.27E−03 |
| | ENDOMEMBRANE SYSTEM | 6 | 6.5 | 2.32E−02 | 4 | 7.6 | 5.24E−03 |
| | PLASMA MEMBRANE | 16 | 20.8 | 2.70E−02 | 16 | 20.3 | 3.57E−02 |
| | MEMBRANE FRACTION | 8 | 10.4 | 3.04E−02 | 7 | 10.1 | 3.56E−02 |
| | SOLUBLE FRACTION | 4 | 5.2 | 9.40E−02 | | | |

The set of 291 low-dose responsive genes was further assigned to subcellular locations, cellular homeostasis functions and signal transduction pathways to provide additional insight into the low dose cellular radiation-response functions. For the subcellular locations (Table 5—Radiation-responsive genes assigned to sub-cellular locations), the majority of genes were mapped to both membrane and cytoplasmic compartments. These included genes integral to the cell membrane (HMGCR SLC1A1 TMP21 DSG2 CD53 GLG1 GGH PTTG1IP C1ORF8 SMBP SPCS2 ARL6IP5 MAN1A1 ENTPD1 MS4A1 CD48 TM9SF2 FAS SSR1 C11ORF15 CKLFSF6 SLC30A1 SORL1 ATP1B3 RPN2 ATP10D ZMPSTE24 PTPRC RPN2 ELOVL5 SSR1 CNIH SLC38A1 SLC35A3 TRAM1 TFRC CD48 CD58 CD164 GALNT7 TXNDC4 TLOC1 LYRIC VDP CGI-100 HIP14 MGC8721 SCAMP1 TEB4 TGOLN2 VMP1 COCH HLA-B, SLC39A6 SLC38A), nuclear envelope (SPCS3 HMGCR SPCS2 GLG1 TMP21 RPN2 TXNDC4) and various organelle endomembrane systems such as the smooth and rough endoplasmic reticulum (HMGCR SART2 SPCS2 ZMPSTE24 MAN1A1 ARL6IP5 SSR1 RCN2 SLC35A3 TMP21 ALG5 SLC1A1 KTN1 TXNDC4 TLOC1 JWA SPTLC1 RPN2 CGI-100), mitochondria (DLD MRPS6 UCP2 SLC25A3 NDUFS1 FLJ10618 C140RF2), golgi apparatus (TMP21 ALG5 MAN1A1 ZMPSTE24 SLC35A3 GLG1 GMH1 TGOLN2 SCAMP1 VDP), and lysosomes (CD63 HEXB GGH LIPA PPT1 CD164 TM9SF2 TFRC CTSZ LAMP2 CTSS).

TABLE 5

| | |
|---|---|
| Integral to membrane | TDE2 HMGCR SLC1A1 TMP21 TPARL SLC39A14 TMEM33 ITM2B |
| | ALG6 MCP DSG2 AGPAT5 CD53 GLG1 GGH GOLPH2 SLC25A3 SPCS3 |
| | PTTG1IP SLC39A8 RNF139 ALCAM LRMP C1ORF8 ATP6V1C2 IFNGR1 |
| | SMBP EIF2AK3 SPCS2 ARL6IP5 MAN1A1 ENTPD1 NUP43 ATP2C1 |
| | SC5DL FLJ10134 ATP11B CD63 SLC12A2 MS4A1 CD48 TM9SF2 TGFBR3 |
| | RSAFD1 FAS TDE1 ITM2A CD38 EBI2 DKFZP564G2022 SSR1 CD83 |
| | RANBP2 ABCC4 ACSL1 C11ORF15 CKLFSF6 CDA08 SLC30A1 SLC9A6 |
| | SORL1 TAP1 SGCE ATP1B3 RPN2 ATP10D ZMPSTE24 STEAP EDELR2 |
| | LRMP LAMP2 ATP6A2 PTPRC PTPRK RNF139 MCP RPN2 LAPTM4A |
| | LEMD3 ELOVL5 ZDHHC17 SSR1 CNIH SLC38A1 SLC35A3 TRAM1 |
| | TFRC CD48 CD58 CD164 GALNT7 TXNDC4 TLOC1 LYRIC VDP CGI- |
| | 100 HIP14 MGC8721 SCAMP1 TEB4 TGOLN2 VMP1 COCH SDFR1 |
| | CAPZB HLA-B HLA-DRA ITGB1 UCP2 PGRMC1 RANBP1 RDX SLC33A1 |
| | SLC39A6 SLC38A |
| Nuclear Envelope- | SLC9A6 SPCS3 RANBP2 LRMP HMGCR ATP11B SPCS2 STEAP LEMD3 |
| Endomembrane | GLG1 TMP21 STCH RPN2 TXNDC4 |
| Endoplasmic | SPCS3 SC5DL EDELR2 HMGCR SART2 GRP58 P4HA1 SLC9A6 P44S10 |
| Reticulum | HSPA5 HSP1A1 LRMP TXNDC5 ALG6 TAP1 EIF2AK3 SPCS2 TRA1 |
| | ZMPSTE24 MAN1A1 ARL6IP5 RCN1 RTN4 SSR1 RCN2 SLC35A3 |
| | TMP21 ALG5 TDE2 SLC1A1 TPARL KTN1 TXNDC4 TLOC1 JWA SEC63 |
| | GRP58 SPTLC1 SGSE SLC9A6 TRA1 TAP1 CANX RPN2 CGI-100 |
| Golgi Apparatus | TMP21 ALG5 MAN1A1 ZMPSTE24 SLC35A3 ZDHHC17 GLG1 GMH1 |
| | TGOLN2 HSP1A1 AKAP1 AKAP11 COPB GALNT1 SCAMP1 EDELR2 |
| | GOLPH2 VDP STEAP |
| Mitochondrion | DLD MRPS6 UCP2 SLC25A3 NDUFS1 FLJ10618 C14ORF2 |
| Lysosome and | CD63 HEXB GGH LIPA PPT1 CD164 TM9SF2 TFRC |
| Endosome Systems | CTSZ LAMP2 CTSS |
| Cytoplasm | SC5DL HMGCR TMP21 ALG5 AKAP9 CD63 SART2 IFI44 LIPA NDUFS1 |
| | G1P2 P4HA1 FUBP1 HSPA5 TXNDC5 TM9SF2 ALG6 UBA52 RTN4 SSR1 |
| | GLG1 GOLPH2 SLC25A3 HEXB SPCS3 DLD GGH PAM PTTG1IP |
| | HSPA1B PPT1 TMSB10 RARS MRPS6 GRP58 C15ORF24 SLC9A6 LRMP |
| | P44S10 EIF2AK3 RPS29 TAP1 TRA1 SPCS2 MAN1A1 HSP1A1 SGCE |
| | ZMPSTE24 ARL6IP5 RCN1 STEAP ZDHHC17 RCN2 NACA HSPA1A |
| | COCH EPRS FLJ10900 HNRPD ELOV5 LOC5681 P5 PTTG1IP |
| | RNASET2 IF144 PCM1 RAD54B UBA52 LOC56851 GIP2 MBNL1 HNRPD |
| | COPB TNFRSF6 FUCA1 HEXB PGRMC1 NARS DDX17 RPS2S RPS29 |
| | RPL5 RPL17 RPL24 RPL37A ACSL3 ACSL1 |
| Unknown | FLJ20696 UNC50 LRBA DJ97N18.2 FLJ10652 FLJ10900 HTGN29 |
| | MGC5306 |

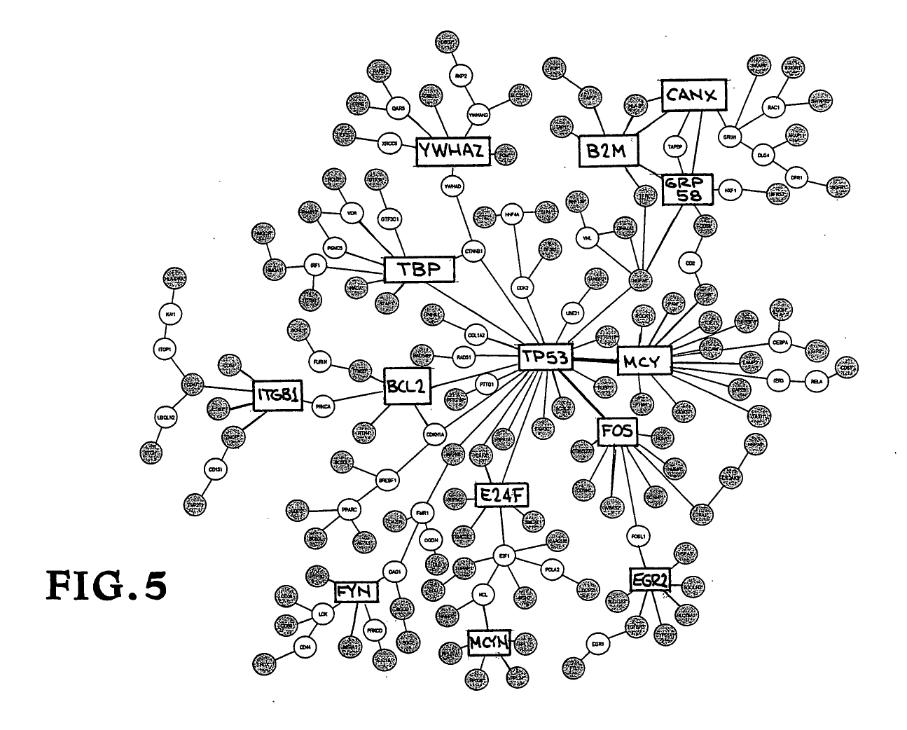

The low-dose responsive genes assigned to homeostasis functions and signal transduction pathways (Table 6A & B—Radiation-responsive genes assigned to cellular homeostasis functions and signal transduction pathways) appear to fall into two categories: membrane-associated and DNA-associated. The genes that mapped to homeostasis (Table 6A—Homeostasis) include those involved with solute transport, cellular energy, metabolism, stress response, and cancer-related functions. The solute-transport genes include those involved with transport of metal ions, sodium and potassium, amino acid, nucleotides, glucose and fatty acids; e.g., SLC1A1 (glutamate), SLC39A14 (zinc ion), SLC25A3 (phosphate), SLC39A8 (zinc), RCN1 and RCN2 (calcium ion), SLC12A2 (sodium chloride/potassium chloride), SLC30 A1 (hydrogen peroxide), SLC9A6 (sodium ion/proton). Energy-associated genes were primarily involved in ATPase functions for small molecule membrane mediated transport, such as ATP1B3, ATP2C1, ATP11B, and ATP10D. Genes associated with cellular metabolism included ARL6IP5 (amino acid); ACSL1 and ACSL3 (fatty acid); MAN1A1 (carbohydrate metabolism), LIPA (lipid metabolism); GALNT7, TXNDC4, RPN1, RPN2 (glycoprotein metabolism), and GLG1 (amino acid hydrolysis). A broad variety of signal transduction pathways were associated with low-dose response (Table 6B—Signal Transduction Pathways) including those associated with cell cycle control, DNA synthesis, cell cycle control, recombination as well as a variety of other membrane-trafficking, signaling, and stress-response pathways (FIG. 5). Implied are chief effectors of the major nodes that include RelA, EGR2, EGR1, E2F1 and E2F2, and TGFBR, through pathways that include the p38 MAPK, SAPK/JNK, JAK/STAT, JAK/AKT, cytokine IL2, and IL4, and NF-kB signaling. Table 7—Examples of radiation-responsive genes that are associated with lymphocyte functions list examples of low dose radiation-responsive genes with lymphocyte-specific functions.

TABLE 6A

| | | |
|---|---|---|
| Solute Transport | Cations & Anions | SLC39A14 SLC25A3 SLC39A6 SLC39A8 SLC12A2 |
| | | SLC30A1 RCN1 RCN2 PAM DLD SLC33A1 SLC16A1 |
| | | SLC9A6 TXNDC4 GRP58 ATP6VOA2 TFRC TM9SF2 |
| | | SLC38A1 SLC1A1 FTL FLJ10618 UBA52 METTL3 |
| | | SWAP70 FLJ22625 DKFZP564K142 SMBP |
| | Amino Acid | ARL6IP5 SLC1A1 JWA UBE4A LAPTM4A |
| | Peptide | SEC3L1 ABCC4 VDP SORL1 SCAMP1 TMP21 NUP43 |
| | Protein | TRAM1 |
| Energy | ATPase Function | ATP1B3 ATP2C1 ATP11B ATP10D ATP6AP2 ATP6V1C2 |
| | for Transport | STCH P44S10 SMC4L1 DLD TRA1 |
| Metabolism | Fatty acid & Lipid | ACSL1 ACSL3 LIPA ELOV5 SORL1 DLD MINPP1 SQLE |
| | | P5 ALG5 ALG6 SIAT1 PTPRC HMMR HMGCR SC5DL |
| | | STCH HIP14 SPTLC1 |
| | Glycoprotein | GALNT1 GALNT7 TXNDC4 RPN1 MAN1A1 RPN2 SGCB |
| | | SGCE DDOST HEXB ALG5 ALG6 |
| | Carbohydrate | MAN1A1 FUCA1 |
| | Protein Synthesis, | GLG1 GGH PAM HSPA1A RARS RPL17 NARS LOC5681 |
| | Modification, | P5 ZMPSTE24 CAPZB RISC NACA HS2ST1 MRPS6 |
| | Stability & | OAZIN USP1 RPS29 RPS28 RPL5 RPL24 RPL37A RNF13 |
| | Degradation | CTSZ PSMD14 PTPRK |
| | | TAP1 UBE4A |
| | RNA & DNA | DDX17 DD5 DHX15 PRPF4B GTF3A SF3B1 PAICS |
| | | DNAJA1 DNAJB9 RPL5 RNASET2 HNRPD TARDBP |
| | | SFRS7 ENPP4 EPRS TEB4 |
| Stress Response | Membrane | GLG1 RTN4 RDX CD63 HEXB GGH LIPA PPT1 CD164 |
| | Structure & Cell | CD48 HSPA1B FLJ10618 TGFBR3 TM9SF2 TFRC TMP21 |
| | Signaling | RANBP2 KPNA2 LAP1B NUP37 JWA MGC14799 CAPZB |
| | | RPA1 FURIN SORL1 CYP5A1 HSPA5 TGOLN2 TRA1 |
| | | UBA52 RCN1 HMMR HNF4A GRP58 FLJ10900 PRDX4 |
| | | VMP1 ZMPSTE24 RANBP2 ROCK1 RCN1 RCN2 SGSE |
| | | TLOC1 CYP51P2 DHRS8 MATR3 HSPA8 PNN CDA08 |
| | | NIPA2 SGCB HMGA1 SQLE TRAM1 |
| Cancer Related | Tumorigenesis, & | FUBP1 ITGB1 MSH2 LYRIC AKAP9 B2M CANX CD38 |
| | Metastasis | CD47 CD48 CD58 CSE1L FUBP1 G1P2 IFNGR1 GRP58 |
| | | HMGA1 HMMR HSPA5 HSPA8 HSPA1A ITGB1 MCP1 |
| | | MS4A1 MSH2 PAM PTPRC PTPRK RDX SEC63 TFRC |
| | | TGFBR3 TNFRSF6 TRA1 UCP2 USP1 CTSZ RNF139 DCN |
| | | SART2 HMGA1 DD5 STEAP |

TABLE 6B

| | |
|---|---|
| Cell Cycle | RPL5 PRKCA JWA MATR3 ZRF1 CDKN1A |
| Control. Survival | MAPK6 TNFRSF6 PTPRC MGC5306 RNASET2 |
| & Apoptosis | GRP58 CSE1L DD5 TXNDC4 SMC2L1 PTMA |
| DNA Repair, | BTAF1 MSH2 CREB3L2 CD38 FUBP1 GTF3A SSR1 |
| Recombination & | CSE1L DSG2 EPRS GTF3A ILF2 NACA PCM1 |
| Replication | RARS RCN2 HSPCA1A HSPA5 RAD54B RANBP2 |
| | SF3B1 POLQ FUBP1 H2AFX MSH2 RAD54B |
| Immune | TGFBR3 ILF2 HLA-DRA GRP58, HLA-B HLA-DRA |
| Response & Cell | CD44 SDFR1 PRDX4 ITGB1 IFNGR1 ITGA4 |
| Signaling | LAMP2 ROCK1 TDE1 TNFRSF6 GIP2 GGH GLG1 |
| | CD48 CD58 CD83 HMGCR PAM TFRC PTPRC |
| | ALCAM CAPZB OAZIN IFI44 SDFR1 CD164 MCP1 |
| | HMMR MS4A1 SART2 CD38 CD53 RNF13 RNF139 |
| | MAPK6 CNIH CTSS PRBP4 AKAP1 AKAP9 |
| | AKAP11 TAP1 EB12 ENTPD1 GIP2 KTN1 |
| Unknown | FLJ20696 UNC50 RPN4 FLJ10525 C6ORF6 TPARL |
| | KIAA0186 KIAA0033 KIAA0102 KIAA0922 |
| | KIAA1815 AF31104 C1ORF8 C11ORF15 CGI-100 |
| | CKLFSF6 COCH DJ97N18.2 FLJ10652 FLJ10900 |
| | GMH1 HTGN29B LOC56851 |

TABLE 7

| Gene ID | Function |
|---|---|
| IFNGR1 | Cytokine Receptor |
| HLA-DRA | Class II Antigen presenter |
| ILF2 | Immune Response |
| LRBA | Trafficking of vesicles |
| HLA-B | Class Antigen presenter |
| LRMP | Developmental regulation |
| CD53 | Signal transduction |
| CD63 | Blood platelet activation factor |
| SART2 | Tumor-rejection antigen |
| MCP | Inactivation of complement |
| ETEA | Resistance to apoptosis |
| CTSS | Degradation of antigenic proteins |
| MS4A1 | B-cell activation |
| CD47 | Signal activation |
| EB12 | EBV Infection of B cells |
| CD58 | Humoral response |
| ENTPD1 | Humoral response |
| ALCAM | Humoral response |
| GIP2 | Immune response |
| CD83 | Humoral response |
| CD48 | Defense response |
| CNIH | Immune response |
| CD164 | Signaling |
| CD59 | Signaling |
| IF144 | Response to virus |

Gene Interaction Networks

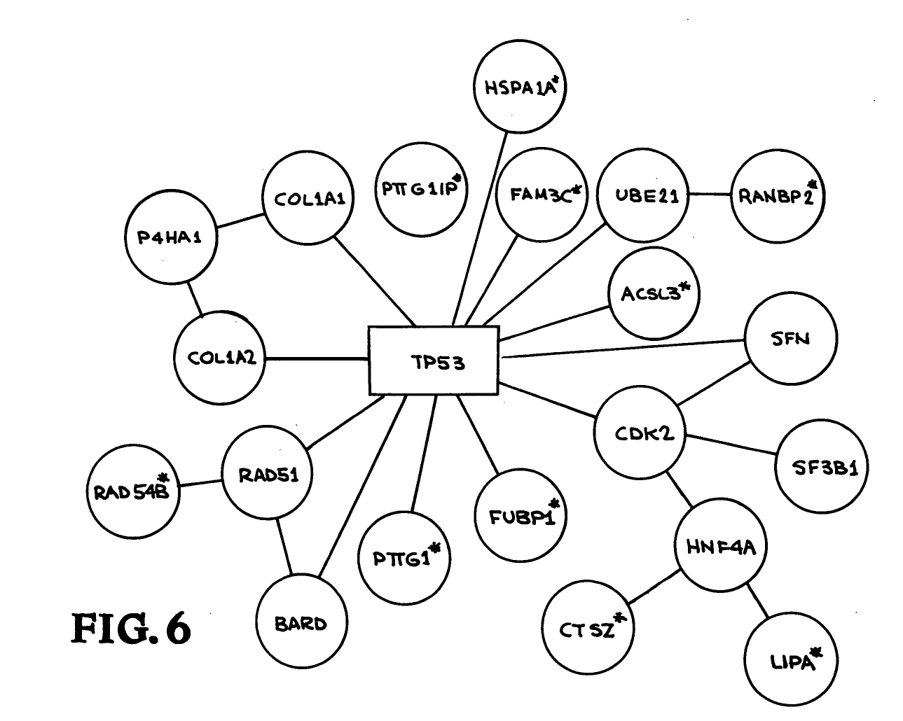

Ingenuity tools were used to construct putative gene interaction networks for the set of 291 low-dose responsive genes. A total of 279 gene associations were identified of which a subset of 111 (focus genes) were specific to the IR-responsive gene set (Table 8—Eight major gene networks in the candidate set of 291 low-dose responsive genes). The 111 focus genes fell into 8 major network groups that were assigned to various top functions with rank scores ranging from 11-21, with 11-17 focus genes each. FIG. 6 is an integrated model for a gene-interaction network for cellular pathways and functions implicated by our low-dose transcript findings. The network connects all of the 8 network groups into a single model using gene associations identified within the literature. A total of 15 central nodes (genes with >5 connections) were identified within this network model. Four of the 15 nodes were for genes identified as responsive in the low dose range. These were Beta-2-microglobulin (B2M) a class I MHC receptor component involved in immune responses, Calnexin (CANX) an integral membrane protein of the endoplasmic reticulum (ER) that plays a role in the regulation of cellular metabolism, Protein disulfide isomerase family A, member 3 (GRP58) that may function as a chaperone and Integrin, beta 1 (ITGB1) a membrane bound protein involved in integrin-mediated signal transduction.

TABLE 8

| | | | | Number of | |
|---|---|---|---|---|---|
| | | | | focus | |
| | | Rank | | genes | |
| Top function per network | | Score | p-value | per network | Example genes |
| 1 | Nervous system development | 21 | | 17 | CANX, GRP58, |
| | and function, Cancer, | | | | IFNGR1, TFRC, |
| | | | | | VDP |
| 2 | Immune Response, tissue | 17 | | 15 | ALCAM, CD48, |
| | development, cell-to-cell | | | | CD58, CD83, |
| | signaling and interaction | | | | TNFRSF6 |
| | (myc node, FIG. 5) | | | | |
| 3 | Tissue development and | 17 | | 15 | CD164, Cyp51A1, |
| | Morphology, gene expression | | | | HMMR, TGFBR3, |
| | (fos node, FIG. 5) | | | | TRA1 |
| 4 | DNA Replication, | 17 | | 15 | BTAF1, CTSS, |
| | Recombination and Repair | | | | HMGA1, |
| | | | | | HMGCR, PCM1 |
| 5 | Lipid Metabolism, nutritional | 15 | | 14 | ACSL1, HLA- |
| | disease | | | | DRA, ITGB1, |
| | | | | | MCP, TMP21 |
| 6 | Cell cycle, DNA Replication, | 14 | | 13 | DCP2, EDD, |
| | Recombination and Repair | | | | H2AFX, HNRPD, |
| | | | | | MSH2 |
| 7 | Cell cycle, DNA Replication, | 11 | | 11 | ACLS3, CTSZ, |
| | Recombination and Repair | | | | FUBP1, RAD54B, |
| | (tp53 node, FIG. 5) | | | | RANBP2 |
| 8 | Cell-to-cell Signaling and | 11 | | 11 | CD38, MAPK6, |
| | ineraction, hematological | | | | MS4A1, OAZIN, |
| | system development | | | | PTPRC |

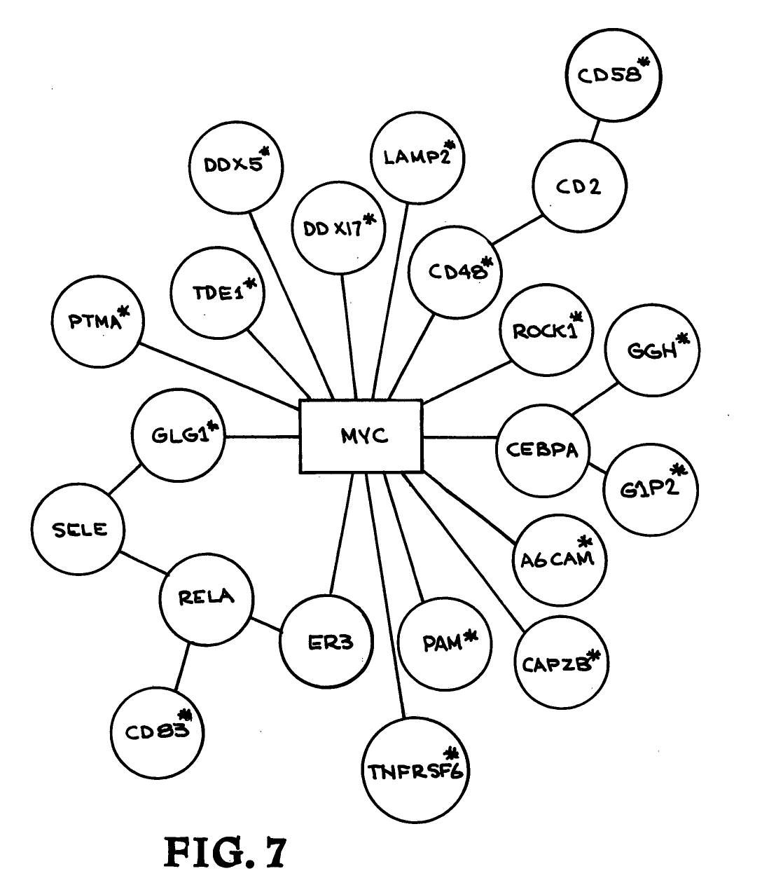

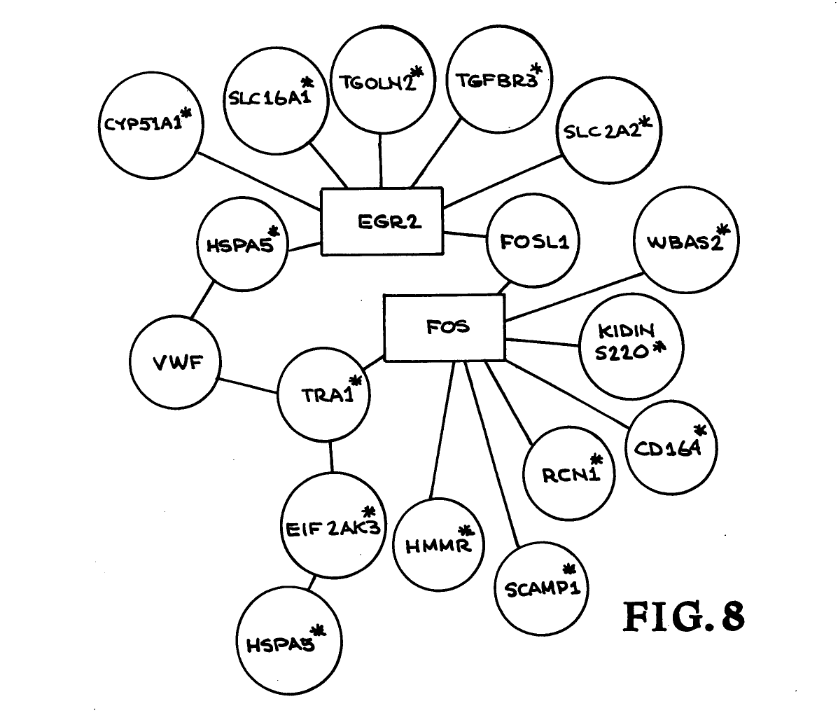

Three nodes were determined to be central to Applicants low dose interaction network based on the number interactions drawn between the responsive genes being >8 connections. FIG. 7 illustrates these three major networks that contain the central nodes for MYC, FOS and TP53, respectively. Ingenuity analyses of the set of consistent 81-gene lists identified 38 focus genes and 2 major networks with rank scores of 18 and 20 and with 11 and 12 focus genes, respectively (supporting material, Figures S9 and S10). The top functions of the two networks from the 81-gene list were associated with maintenance of cellular homeostasis and signal transduction pathways. FIG. 6 shows were the 81 gene of the consistent set map onto the composite interaction network that was based on the 291-gene set.

Membrane Pathways of LDIR Response

Bioinformatic analyses of the radiation-responsive data sets provides mechanistic insights into the low dose responses of irradiated cells that could impact cell fate. These analyses identified several distinct aspects of the low-dose IR response: (1) involvement of several subcellular compartments and membrane based processes; (2) modulation of diverse homeostasis functions to include several cellular signaling pathways, stress response, DNA repair, tumorigenesis and metastases; (3) assignment of candidate genes within a 8 gene-interaction networks that were joined to make an model of low-dose response (FIG. 7). Applicants' detection of multiple gene transcripts encoded genes with diverse functions (membrane signaling and damage sensing, small molecule transport, immune modulation, cell-cell communications, and cellular metabolism) and signal transduction. Membrane functions were the dominant group and implicated the involvement of cellular and nuclear membranes, mitochondria, ER and lysosomes as the major cellular functions associated with the low dose in the low dose radiation response (tables 5 and 6) that fell into significant membrane-related GO categories (Table 4). Analyses using EASE, GO and Intenguity tools provided supporting information regarding the different functions, pathways and subcellular organelles modulated by low doses of IR. Previous studies have shown that high levels of UV-irradiation, IR and electromagnetic radiation can lead to both irreversible as well as reversible structural and functional changes within cells and organelles (for a review see Somosy Z, 2000), to Applicants knowledge this is the first comprehensive genome-scale analyses of such effects of IR low doses associated with a large number of intracellular organelle effects. Combined these findings contribute to a better understanding of the general biochemical and cellular mechanisms modulated by low dose exposures that may be important for understanding non-linear low-LET radiation biological effects.

Inter/intra organelle membrane transport proteins (SLC family, TFRC etc.) play an important role in affecting cellular homeostasis in response to stress. Mercier et al emphasized membrane bound and other subcellular proteins involved in mitochondrial processes differently expressed by low dose IR exposures in yeast. The outer mitochondrial membrane has important functions in the metabolic coupling between the cytosol and mitochondria. The transcript modulation of several mitochondrial proteins in Applicants study suggests that mitochondria are involved in the low dose response. The translocase of the outer mitochondrial membrane complex (TOM) and the translocase of the inner mitochondrial membrane TIM23 complex of the inner membrane were identified in the low dose response. Studies with the TOM machinery show that it acts as a receptor complex to allow mitochondrial proteins to enter the organelle. Other transport examples include a large number of ion channels such as the solute carrier family of proteins that serve to facilitate the passage of selected solutes across the lipid barrier. Examples identified were SLC1A1 (glutamate), SLC39A14 (zinc ion), SLC25A3 (phosphate), SLC39A8 (zinc), RCN1 and RCN2 (calcium ion), SLC12A2 (sodium chloride/potassium chloride), SLC30A1 (hydrogen peroxide), SLC9A6 (sodium ion/proton).

Interactions between signaling pathways and the cytoskeleton were found within Applicants low dose responsive gene set. Annexins (ANX family) are members of a multigenic family of Ca²⁺-dependent phospholipid-, membrane- and cytoskeleton-binding proteins that have also been implicated in the regulation of the inflammatory response, the structural organization of membranes, ion flux across membranes for signaling and for disease. Applicants also identified transcripts encoding CD48, CD58 and CD164, which are membrane bound receptors (among the 80 low dose responsive genes) and cell membrane glycoproteins and members of the cell-organelle interaction. Signal transduction is in fact a three-dimensional exercise in cell biology and interactions between signaling pathways and the cytoskeleton are functionally important. Previous studies have shown distinct cytoskeletal changes in the organization and composition of the glycolipids, modified activity of membrane domains of the major cellular organelles. Although it is well established that high doses of IR induce changes in cell shape, cell surface micromorphology and the subcellular organelles, Applicants findings suggest that these structural changes and signaling functions are also important for cellular low dose responses.

### Networks of LDIR Response

Network nodes identified interesting areas of biology that may be important for assessing biochemical pathways modulated by low doses of IR. Based on the number of connections between individual genes Applicants identified nodes with the largest number of links between MYC, TP53 and FOS associated functions. The nodes TP53, FOS and MYC identified associations with genes participating and or influencing the cell cycle G1/S and G2/S check points, DNA damage response and Repair, apoptosis and death receptor signaling as well as those that respond to immune signaling and oxidative stress. Importantly, these three proteins are involved in cellular functions through transcriptional mechanisms such as cell cycle control, metastasis, apoptosis and proliferation. Most of what Applicants know about these genes is related to their functions in cancer biology. These proteins are also associated with negative cellular outcomes that are associated with high-dose IR exposures. Other low dose studies have previously implicated the importance of these three key pathways for modulating cellular outcomes such as cell cycle arrest, DNA repair, heat shock, cytokines and cellular proliferation.

TP53 is a central transcription factor activated in response to a variety of known cellular stresses, including DNA damage, mitotic spindle damage, heat shock, metabolic changes, hypoxia, viral infection, and oncogene activation. These processes were also identified as associated with TP53 in the low dose networks identified. The primary genes associated with this specific network implicate the following transcriptionally-related genes post exposure: the Far upstream element-binding protein (FUBP1), which is known to regulate MYC protooncogene expression, the Family with sequence similarity 3 member C (FAM3C) a novel family of cytokines, the heat shock 70 protein (HSPA1A), and the Pituitary tumor-transforming gene 1 protein-interacting protein (PTTG1IP) transcription factor. Other genes associated with this node grouping included proteolytic and metabolic genes (LIPA; CTSZ; P4HA1 and SF3B1). Two other outlying genes within the group included RANBP2 a small GTP-binding protein of the RAS superfamily and RAD54B a member of the SNF2/SWI2 superfamily that is involved in the recombinational repair of DNA damage. Genes TP53 is known induce a number genes involved in cell cycle control, apoptosis and cellular proliferation in response to ionizing radiation at higher doses. Most of these genes such as DDB2, PCNA, and P21 were not identified within this study indicating an alternative TP53 cellular low dose responsive pathway.

Applicants' results also implicate MYC in the low dose response by identifying 11 transcripts directly associated with this node and 3 indirectly. Several of these genes are oncogenes themselves and/or directly involved in immune functions such as apoptosis. Noteworthy, a number of the transcripts encode membrane bound proteins. Genes directly linked to the MYC node with diverse function included Peptidylglycine alpha-amidating monooxygenase (PAM) a multifunctional metabolism protein; Golgi sialoglyco protein (GLG1) a conserved membrane sialoglycoprotein found within the Golgi of most cells; Tumor differentially expressed 1 (TDE1) which is overexpressed in lung tumors, interestingly TDE1 contains a characteristic transmembrane domain, and it has several potential phosphorylation sites; Lysosomal membrane glycoprotein (LAMP2) that are responsible for the degradation processes; Cap protein (CAPZB, also known as Actin beta) an actin-binding protein and Dead/h box17 (DDX17) a DEAD box (asp-glu-ala-asp/his) RNA helicases involved in that may alter protein-RNA interactions as a splicing regulatory factor. MYC node associated transcripts with immune/lymphocyte functions included the following: ROCK1 a downstream effector of Rho, involved remodeling of the actin cytoskeleton, CD48 an activation-associated cell surface glycoprotein expressed primarily in mitogen-stimulated human lymphocytes; TNF receptor form 6 (TNFRSF6 also known as CD95 or FAS) is a oncogene that requires interactions on the cell surface and functional MYC to induce apopotosis; Prothymosin-alpha (PTMA) important for immune function and may provide anti-apoptotic functions; Activated leukocyte cell adhesion molecule (ALCAM also known as CD6) is a receptor thought to be involved in cell adhesion interactions. Other indirectly associated transcripts include CD83 an immune-related adhesion receptor; the interferon-induced protein (G1P2) and Gamma-glutamyl hydrolase (GGH) a metabolic enzyme.

The MYC family members are known primarily as transcription factors that are known to induce transcripts for cyclins such as D1, E and A and cdc25A in response to high doses of IR. At low doses Applicants find primarily membrane bound MYC-associated functions associated with cytoskeletal, metabolism, and various immune functions. This set of transcripts included a strong apoptotic associated gene TNFRSF6. Interestingly, the TNFRSF6 transcript is also clearly associated with high-dose IR functions) and different qualities of ionizing radiation (DING et al 2005 HZE-particle irradiation). Two genes thought to be associated with anti-apoptotic functions were also identified as LDIR responsive CD83 and PTMA. How these genes lead to a specific cell fate outcome at low dose remains unknown. Oncogenes such as c-myc have been previously involved in low dose effects. Applicants' results also suggest that the N-MYC node (FIG. 6), a member of the MYC family, also functions at low doses, but as a primary regulator of cell growth by stimulating genes functioning in ribosome biogenesis and protein synthesis (FIG. 6).

Applicants' findings also implicate FOS in the low-dose response. The FOS gene family consists of 4 members that encode leucine zipper proteins that dimerize with proteins of the transcription factor JUN family, thereby forming the transcription factor complex activating protein AP-1. The transcripts associated with this node were the following: Reticulocalbin 1 (RCN1) a calcium-binding protein located in the lumen of the ER; Hyaluronan-mediated motility receptor (HMMR) which interacts with RHAMM to respond to wound healing; Tumor rejection antigen 1 (TRAL) a protective gene with multiple functions related to chaperonens and production of cytokines IL12 and TNFA; Secretory carrier membrane protein 1 (SCAMP1) a putative transmembrane/leucine zipper-like containing protein and CD164 a sialomucin membrane-associated protein that can be cytoprotective.

Low Dose Cellular Implications

Understanding the biological consequences of exposures to low-dose radiation is becoming increasingly important for humans and other organisms as greater exposures to ionizing radiation occur from new man-made sources and space travel. The cellular response to IR consists of an integrated network of protein signaling and transcriptionally regulated pathways. In this study, Applicants focused on the mathematical description of transcriptional changes as result of varying IR doses. High-density microarray data was analyzed using a newly developed gene-list matching method and well known self-organized map approach. All the methods consistently detect a transition in the cellular response from low to high doses of IR in the 10-15 cGy range. Gene Ontology of the genes in low and high doses is also indicative of the possible functional differences. The findings in this study elucidated parts of the intricate network of genes that are involved in the IR-response.

Identification and detection of a low dose radiation threshold is extremely important in the area of biological dosimetric studies. In the absence of direct data, the biological effects of low-dose radiation are currently estimated by extrapolating from the biological effects of high-dose radiation. This extrapolation is embodied in the linear non-threshold model, which postulates that low-dose radiation is just as harmful per gray as high-dose radiation; thus any dose no matter how small is potentially harmful and has been a subject to considerable discussion and controversy. However, the biological effects of low-dose radiation are considerably more complex than predicted by the linear non-threshold model, and some data seem to support other models. For example, a threshold based model can be developed to postulate that low-dose radiation is harmless below a certain level, such as the one detected in this work.

High-dose IR causes significant biochemical, physiological and genetic damage to cells and tissues that can lead to cell death especially in the radiosensitive cell types such as lymphocytes as well as certain hematopoietic and gastrointestinal cell types, to increased genetic damage, and to late effects such as fibrosis and cancer. Radiation toxicity is induced by direct cellular damage from charged particles and from induced toxic reactive oxygen (ROS) and nitrogen species (NOS) that damage targeted cells and possibly non-irradiated neighboring bystander cells; the latter has special importance in the low dose range. The low-dose response included multiple homeostasis genes associated with broad aspects of metabolism, and stress response that appeared differ greatly from those transcripts found at higher IR exposures. Many of the genes Applicants identified as modulated by LDIR had membrane associations and the networks indicated a high number of immune functions. The transcripts most commonly seen at high dose and associated with cell cycle arrest (for example p21 and DDB2) and apoptosis (CASP genes) were not seen within this low dose modulated gene study. A common finding across Applicants low-dose studies is that heat shock, transcription and cell cycle responses are transcriptionally modulated after low doses of radiation, an observation that could potentially impact low-dose risk assessment modeling. Interestingly, there was a substantial number of genes typically associated with carcinogenesis and metastasis as indicated by the GO searches and the Ingenuity interaction networks. Related genes included FUBP1 ITGB1 MSH2 LYRIC AKAP9 B2M CANX CD38 CD47 CD48 CD58 CSE1L FUBP1 G1P2 IFNGR1 GRP58 HMGA1 HMMR HSPA5 HSPA8 HSPA1A ITGB1 MCP1 MS4A1 MSH2 PAM PTPRC PTPRK RDX SEC63 TFRC TGFBR3 TNFRSF6 TRAL UCP2 USP1 CTSZ RNF139 DCN SART2 HMGA1 DD5 STEAP. These findings highlight the diverse functional areas that could have an affect on the cellular outcome after LDIR.

While the invention may be susceptible to various modifications and alternative forms, specific embodiments have been shown by way of example in the drawings and have been described in detail herein. However, it should be understood that the invention is not intended to be limited to the particular forms disclosed. Rather, the invention is to cover all modifications, equivalents, and alternatives falling within the spirit and scope of the invention as defined by the following appended claims.

The invention claimed is:

1. A method of characterizing exposure to ionizing radiation, comprising the steps of:
selecting a set of biomarkers for characterizing exposure to ionizing radiation, and
using said set of biomarkers for characterizing exposure to ionizing radiation.

2. The method of characterizing exposure to ionizing radiation of claim 1 wherein said step of selecting a set of biomarkers for characterizing exposure to ionizing radiation comprises utilizing a set of 420 oligonucleotide probes for human genes capable of discerning past exposure to different doses of ionizing radiation.

3. The method of characterizing exposure to ionizing radiation of claim 1 wherein said step of selecting a set of biomarkers for characterizing exposure to ionizing radiation comprises groupings of genes that represent candidate panels of mRNA biomarkers that represent genes that are dose specific over a range of fourteen radiation doses including no exposure baseline data for these genes.

4. The method of characterizing exposure to ionizing radiation of claim 1 wherein said step of selecting a set of biomarkers for characterizing exposure to ionizing radiation comprises groupings of genes that represent candidate panels of mRNA biomarkers of which a subset represents genes that are a robust panel that discriminate between low dose exposure and no exposure.

5. The method of characterizing exposure to ionizing radiation of claim 1 wherein said step of selecting a set of biomarkers for characterizing exposure to ionizing radiation comprises groupings of genes that represent candidate panels of mRNA biomarkers of which a subset represents genes that are a robust panel that is validated across multiple individuals.

6. The method of characterizing exposure to ionizing radiation of claim 1 wherein said step of selecting a set of biomarkers for characterizing exposure to ionizing radiation comprises groupings of genes that represent candidate panels of mRNA biomarkers of which a subset represents genes that are dose specific over a range of fourteen radiation doses including no exposure baseline data for these genes; a robust panel that discriminate between low dose exposure and no exposure, between low and high-dose exposure; or a robust panel that is validated across multiple individuals.

7. The method of characterizing exposure to ionizing radiation of claim 1 wherein said step of using said set of biomarkers for characterizing exposure to ionizing radiation comprises using molecular techniques for characterizing exposure to ionizing radiation.

8. The method of characterizing exposure to ionizing radiation of claim 1 wherein said step of using said set of biomarkers for characterizing exposure to ionizing radiation comprises using LRT-PCR or other method for analyzing RNA levels for characterizing exposure to ionizing radiation.

9. The method of characterizing exposure to ionizing radiation of claim 1 wherein said step of using said set of biomarkers for characterizing exposure to ionizing radiation comprises using DNA test strips for characterizing exposure to ionizing radiation.

10. The method of characterizing exposure to ionizing radiation of claim 1 wherein said step of using said set of biomarkers for characterizing exposure to ionizing radiation comprises using Luminex for characterizing exposure to ionizing radiation.

11. The method of characterizing exposure to ionizing radiation of claim 1 wherein said step of using said set of biomarkers for characterizing exposure to ionizing radiation comprises using microarrays for characterizing exposure to ionizing radiation.

12. The method of characterizing exposure to ionizing radiation of claim 1 wherein said step of using said set of biomarkers for characterizing exposure to ionizing radiation comprises using molecular techniques for characterizing exposure to ionizing radiation that are capable of measuring single or multiple combinations of transcript and protein changes for all or subset panels of the gene probes.

13. A method of characterizing exposure to ionizing radiation, comprising the steps of:
selecting a set of biomarker genes for characterizing exposure to ionizing radiation, and
using said set of biomarker genes for characterizing exposure to ionizing radiation.

14. The method of characterizing exposure to ionizing radiation of claim 13 wherein said step of selecting a set of biomarker genes for characterizing exposure to ionizing radiation comprises utilizing a set of 420 oligonucleotide probes for human genes capable of discerning past exposure to different doses of ionizing radiation.

15. The method of characterizing exposure to ionizing radiation of claim 13 wherein said step of selecting a set of biomarker genes for characterizing exposure to ionizing radiation comprises groupings of genes that represent candidate panels of mRNA biomarkers that represent genes that are dose specific over a range of fourteen radiation doses including no exposure baseline data for these genes.

16. The method of characterizing exposure to ionizing radiation of claim 13 wherein said step of selecting a set of biomarker genes for characterizing exposure to ionizing radiation comprises groupings of genes that represent candidate panels of mRNA biomarkers of which a subset represents genes that are a robust panel that discriminate between low dose exposure and no exposure.

17. The method of characterizing exposure to ionizing radiation of claim 13 wherein said step of selecting a set of biomarker genes for characterizing exposure to ionizing radiation comprises groupings of genes that represent candidate panels of mRNA biomarkers of which a subset represents genes that are a robust panel that is validated across multiple individuals.

18. The method of characterizing exposure to ionizing radiation of claim 13 wherein said step of selecting a set of biomarker genes for characterizing exposure to ionizing radiation comprises groupings of genes that represent candidate panels of mRNA biomarkers of which a subset represents genes that are dose specific over a range of fourteen radiation doses including no exposure baseline data for these genes; a robust panel that discriminate between low dose exposure and no exposure, between low and high-dose exposure; or a robust panel that is validated across multiple individuals.

19. The method of characterizing exposure to ionizing radiation of claim 13 wherein said step of using said set of biomarker genes for characterizing exposure to ionizing radiation comprises using molecular techniques for characterizing exposure to ionizing radiation.

20. The method of characterizing exposure to ionizing radiation of claim 13 wherein said step of using said set of biomarker genes for characterizing exposure to ionizing radiation comprises using LRT-PCR or other method for analyzing RNA levels for characterizing exposure to ionizing radiation.

21. The method of characterizing exposure to ionizing radiation of claim 13 wherein said step of using said set of biomarker genes for characterizing exposure to ionizing radiation comprises using DNA test strips for characterizing exposure to ionizing radiation.

22. The method of characterizing exposure to ionizing radiation of claim 13 wherein said step of using said set of biomarker genes for characterizing exposure to ionizing radiation comprises using Luminex for characterizing exposure to ionizing radiation.

23. The method of characterizing exposure to ionizing radiation of claim 13 wherein said step of using said set of biomarker genes for characterizing exposure to ionizing radiation comprises using microarrays for characterizing exposure to ionizing radiation.

24. The method of characterizing exposure to ionizing radiation of claim 13 wherein said step of using said set of biomarker genes for characterizing exposure to ionizing radiation comprises using molecular techniques for characterizing exposure to ionizing radiation that are capable of measuring single or multiple combinations of transcript and protein changes for all or subset panels of the gene probes.
